(19) United States
(12) Patent Application Publication
(10) Pub. No.: US 2016/0264757 A1
(43) Pub. Date: Sep. 15, 2016

(54) ANTIOXIDANTS FOR PRODUCING LOW-EMISSION PUR SYSTEMS

(71) Applicant: Evonik Degussa GmbH, Essen (DE)

(72) Inventors: Michael Krebs, Dusseldorf (DE); Roland Hubel, Essen (DE)

(21) Appl. No.: 15/016,503

(22) Filed: Feb. 5, 2016

(30) Foreign Application Priority Data
Mar. 10, 2015 (EP) 15 158 424.0

(51) Int. Cl. C08K 5/134; C08G 18/08; C08K 5/1535; C08G 18/48; C09K 15/32; C08J 9/00; C08K 5/526; C08K 5/527; C09K 15/08; C07C 69/732; C08G 18/76

(52) U.S. Cl. CPC C08K 5/1345; C07C 69/732; C08G 18/14; C08G 18/0838; C08K 5/1535; C08G 18/48; C08G 18/7621; C08J 9/0023; C08K 5/526; C08K 5/527; C09K 15/08; C09K 15/322; C08G 2101/0083

(57) ABSTRACT

A compound of the formula (I)

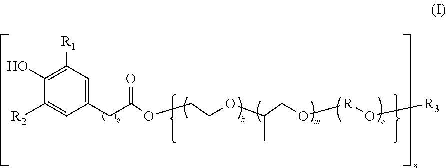

in which
R is CH₂—CH(R^{I}), CH(R^{II})—CH(R^{II}), CH₂—C(R^{II})₂, C(R^{II})₂—C(R^{II})₂,

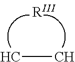

CH₂—CH—CH₂—R^{IV}, C₆H₆—CH—CH₂, C₆H₆—C(CH₃)—CH₂, where
R^{I} is C₂ to C₂₄ alkyl radical or alkene radical,
R^{II} is C₂ to C₂₄ alkyl radical or alkene radical,
R^{III} is C₃ to C₆ alkyl radical, which is arranged linearly, and
R^{IV} is OH, Cl, OCH₃, OCH₂—CH₃, O—CH₂—CH═CH₂, O—CH═CH₂ molecule residue of epoxidized fats or oils,
R₁ and R₂ independently of one another are C₁-C₈ alkyl, cyclopentyl or cyclohexyl, especially tert-butyl,
R₃ is an n-valent radical of C₁-C₃₀-alkyl,
R₄ is hydrogen, an n-valent radical of C₁-C₃₀ alkyl, which is optionally interrupted by one or more groups —NR₅— or (where n=1-12) is an n-valent radical of C₅-C₁₂ cycloalkyl,
R₅ independently at each occurrence is hydrogen or methyl or —C_{q}H_{2q}.

### RELATED APPLICATION DATA

The present application hereby claims priority to European Application No. EP 15 158 424.0 filed Mar. 10, 2015, which is incorporated herein by reference in its entirety.

### FIELD

The present invention is situated within the field of antioxidants and also within the field of plastics, more particularly of polyurethanes. It concerns, in particular, new antioxidants, antioxidant mixtures, and polyurethane systems, especially polyurethane foams.

### BACKGROUND

Antioxidants are compounds of various chemical structures and are intended to inhibit or prevent unwanted alterations, caused by oxygen exposure and other oxidative processes, in the substances under protection.

They are required in plastics, for example, for protection from aging, since synthetic polymers are subject fundamentally to oxidation by oxygen, and impurities often present in small amounts may promote the oxidation process. This oxidation can lead to detrimental changes to the mechanical properties of the polymer in question, and hence of the component in which the polymer is used. This may result ultimately in an unwanted deterioration in function. In order to prevent or at least inhibit such oxidation processes, therefore, antioxidants are used, examples being sterically hindered phenols.

This situation may also apply to the production of polyurethane systems, such as polyurethane coatings, polyurethane adhesives, polyurethane sealants, polyurethane elastomers or, in particular, polyurethane foams/foam materials, for example.

Polyurethane systems, such as polyurethane foams in particular, are used across a wide variety of sectors, by virtue of their outstanding mechanical and physical properties. One particularly important market for a wide variety of types of PU foams (=i.e. polyurethane foams), such as conventional flexible foams based on ether and ester polyol, cold-cure foams (frequently referred to also as HR foams), rigid foams, integral foams and microcellular foams, and also foams with properties situated between these classifications, such as semi-rigid systems, for example, is that of the automotive and furniture industries. For instance, rigid foams are used as roof liner, ester foams as interior door trim and also for die-cut sun visors, cold-cure and flexible foams are used for seat systems and mattresses.

There has been no lack of attempts to date to provide antioxidants which are effective in particular with a view to the stabilization of polymers that are susceptible towards oxidative, thermal or light-induced degradation.

For example, hydroxyphenylcarboxylic esters can be used as antioxidants. There are numerous known compounds from the class of hydroxyphenylcarboxylic esters. They can be prepared, for example, by transesterification using suitable catalysts. Transesterification processes of this kind are described in, for example, U.S. Pat. No. 4,716,244, U.S. Pat. No. 5,481,023, U.S. Pat. No. 5,563,291, EP-A-1292560. EP-A-608089 describes esters of the above-stated structural type that comprise polyethylene glycol groups. They too can be prepared by transesterification, in which case the known strongly basic catalysts are recommended, such as alkali metals and also alkali metal amides, alkoxides, hydroxides and (bi)carbonates.

These and other known antioxidants are useful, but do not in every respect satisfy the exacting requirements asked of an aging inhibitor when the stabilization concerned is that of synthetic polymers, especially with regard to lifetime, water absorption, sensitivity to hydrolysis, process stabilization, color properties, volatility, migration characteristics, compatibility and improvement in protection with respect to light.

Consequently there still exists an ongoing need for possibilities for the stabilization of synthetic polymers that are susceptible to oxidative, thermal and/or light-induced degradation.

### SUMMARY

The specific problem addressed by the present invention against this background was that of providing antioxidants which are suitable for the stabilization of synthetic polymers, especially with a view to the provision of corresponding polyurethanes, preferably of polyurethane foams.

Entirely unexpectedly it has now been found that a specific group of compounds of the type of the hydroxyphenylcarboxylic esters do justice to this objective.

### DETAILED DESCRIPTION

A subject of the present invention is a compound of the formula (I)

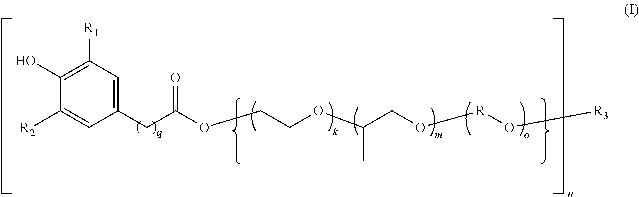

in which
R is CH₂—CH(R^{I}), CH(R^{II})—CH(R^{I}), CH₂—C(R^{II})₂, C(R^{II})₂—C(R^{II})₂,

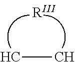

CH₂—CH—CH₂—R^{IV}, C₆H₆—CH—CH₂, or C₆H₆—C(CH₃)—CH₂, where
R^{I} is C₂ to C₂₄ alkyl radical or alkene radical, which may be linear or branched,
R^{II} is C₂ to C₂₄ alkyl radical or alkene radical, which may be linear or branched,
R^{III} is C₃ to C₆ alkyl radical, which is arranged linearly, and
R^{IV} is OH, Cl, OCH₃, OCH₂—CH₃, O—CH₂—CH═CH₂, O—CH═CH₂, molecule residue of singly or multiply epoxidized fats or oils as mono-, di-, and triglycerides, or molecule residue of singly or multiply epoxidized fatty acids or their C₁-C₂₄ alkyl esters,
R₁ and R₂ independently of one another are straight-chain or branched C₁-C₈ alkyl, cyclopentyl or cyclohexyl, especially tert-butyl,
q is 1, 2 or 3, preferably 2 or 3, especially 2,
n is an integer from 1 to 30, preferably an integer from 1 to 10, advantageously 1, 2, 3, 4, 5 or 6, e.g. 1, 2, 3 or 4, especially 1,
R₃ is an n-valent radical of linear or branched C₁-C₃₀ alkyl, preferably C₁-C₁₀ alkyl, C₂-C₃₀ alkylene, interrupted in each case optionally by one or more oxygen atoms, or (where n=1-12) is an n-valent radical of C₅-C₁₂ cycloalkyl, or a radical R₄—[NR₅—C_{q}H_{2q}—]ₚ,
R₄ is hydrogen, an n-valent radical of linear or branched C₁-C₃₀ alkyl, which is optionally interrupted by one or more groups —NR₅— or (where n=1-12) is an n-valent radical of C₅-C₁₂ cycloalkyl,
R₅ independently at each occurrence is hydrogen or methyl or —C_{q}H_{2q}—, preferably hydrogen, and
p corresponds to the number of [NR₅—C_{q}H_{2q}—] groups that produces n radicals —C_{q}H_{2q}— per molecule,
k is an integer between 0 and 50, preferably between 10 and 30,
m is an integer between 0 and 50, e.g. 1-40, and
o is an integer between 0 and 50, preferably between 0 and 30, especially 0,
where (k+m+o)>10.

The problem addressed by the present invention is solved by this subject. It permits very effective stabilization of synthetic polymers against oxidative, thermal or light-induced degradation. This subject has proved to be particularly valuable and efficacious particularly in the context of the provision of polyurethane systems, preferably polyurethane foams, especially free-rise flexible polyurethane (slabstock) foams. The invention enables overall a substantial improvement to be achieved in the sustained retention of the processing and performance features of polyurethane systems, especially PU foams.

If in the formula (I) k, m, o>0 or k, m>0 and at the same time o=0, the sequence of the monomer units ethylene oxide, propylene oxide and (R-oxide) in the individual polymer chains 1 to n is arbitrary, and k, m and o represent average values. Moreover, the individual units (EO), (PO) and (RO) can be bonded to one another either in the form of blocks, in strict alternation or in the form of gradients. In the form of gradients means that in the individual chain there is a gradient in the distribution of the (BO), (PO) and (RO) units along the chains.

Hydroxyphenylcarboxylic esters are sterically hindered phenols. From mechanistics studies it is known that the functional group within the hydroxyphenylcarboxylic esters that counteracts the oxidative degradation of polymers is the sterically hindered phenol unit.

All the more surprising is the fact that the mass fraction of phenol units in the compounds of the formula (I) of the invention is smaller than in existing compounds of the hydroxyphenylcarboxylic ester type and that in spite of this an antioxidant effect (especially with regard to the stabilization of synthetic polymers, such as preferably polyurethane systems) is achieved that in fact exceeds the effect of known hydroxyphenylcarboxylic esters. The real expectation would have been that a greater amount of the compounds of the formula (I) of the invention would have been necessary in order to be able to achieve an antioxidative effect even only comparable to that of the known hydroxyphenylcarboxylic esters. The opposite, however, can be observed.

A further wholly unexpected advantage of the compounds of the formula (I) of the invention is, moreover, that the use of the compounds of the invention permits the provision of polyurethane systems, more particularly PU foams, which exhibit unexpectedly good emissions characteristics. The performance features of polyurethane systems, more particularly PU foams, are in fact improved accordingly.

Exemplary compounds of the formula (I) are the compounds Ia and Ib shown below.

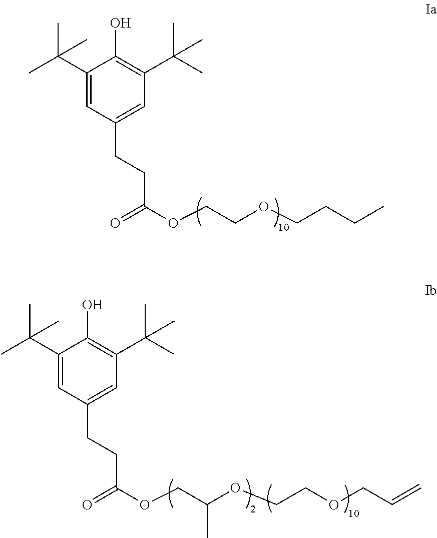

The inventive use of the aforementioned compounds Ia and Ib corresponds to one preferred embodiment of the invention.

A further subject of this invention is a synthetic polymer, preferably polyurethane system, more particularly PU foam, comprising at least one compound of the formula (I).

Such polymers, especially polyurethane systems, are particularly insensitive to aging and particularly oxidation-resistant, and, more particularly, such foams exhibit unexpectedly good emissions characteristics. The influencing of the emissions characteristics of synthetic polymers, especially polyurethane (foam)s, is of great significance.

Thus, for example, in the production and the subsequent use of polyurethane systems, such as foams in particular, the release of emissions and fogging are problem factors. Fogging is understood as the emission of compounds which may subsequently condense, such as in a vehicle interior, for example, on the windscreen, to form a usually hazy covering, for example.

Many consumers are therefore somewhat critical of polyurethane systems and in some cases even have health concerns, despite the objective lack of any justification for health concerns, as demonstrated by results of noxious substance measurements. From both the consumer side and the industry side, therefore, there is an ongoing desire for polyurethane systems of this kind with the smallest possible release of emissions.

As part of this invention it has now been found that the compounds of the formula (I) of the invention permit the provision of polyurethane systems, especially PU foam, with minimal release of emissions and minimal fogging, as compared with polyurethane systems in which conventional antioxidants have been used.

A proven test method for assessing emissions that is established in the market is, for example, the DaimlerChrysler testing instructions of VDA 278: “Thermodesorptionsanalyse organischer Emissionen zur Charakterisierung nichtmetallischer KFZ-Werkstoffe” [Thermodesorption analysis of organic emissions for characterizing non-metallic vehicle material] of October 2011. The figure for the emissions of volatile compounds is referred to below as VOC (VOCs=Volatile Organic Compounds). The value for the emissions of condensable compounds, corresponding to fogging, is referred to below as fog value. Appropriate methods for the determination of VOC and fogging are described with precision in the examples section.

The use of the compounds of the formula (I) of the invention makes it possible, advantageously, to produce polyurethane systems, especially PU foams, which have very low values for volatile organic (VOC) and condensable (fogging) compounds. It has been possible to achieve values of <100 ppm for VOC and of <250 ppm for fogging. The values for VOC and fogging may be determined in particular by means of thermodesorption analysis. Using the compounds of the formula (I) of the invention makes it possible, furthermore, advantageously, to produce polyurethane systems, especially PU foams, which are particularly low in odor. Using the compounds of the formula (I) of the invention makes it possible, additionally, advantageously, to produce polyurethane systems, especially PU foams, which are particularly aging-resistant. Another advantage of using the compounds of the formula (I) is that they can be utilized without complication in existing production operations and on existing production lines.

The compounds of the formula (I) of the invention can be prepared by any suitable process which is common knowledge within the field of the preparation of compounds of this type, including various esterification processes. Three examples of such processes are elucidated exemplarily in the reaction schemes below, and are identified respectively as process A, process B and process C.

### 1) Process A

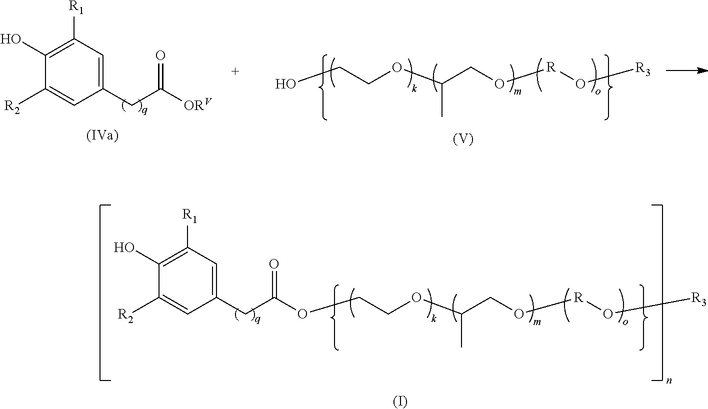

### 2) Process B

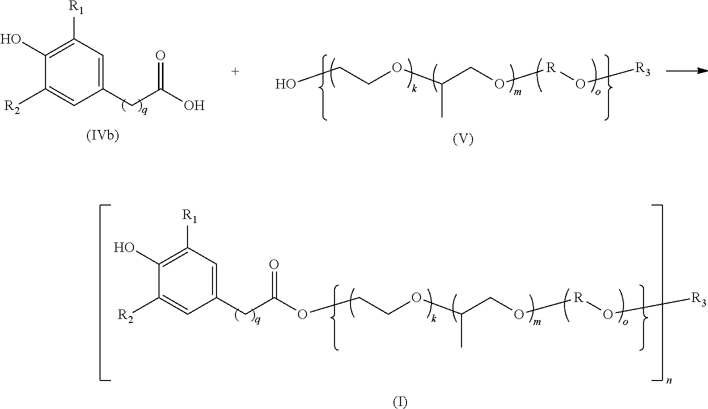

### 3) Process C

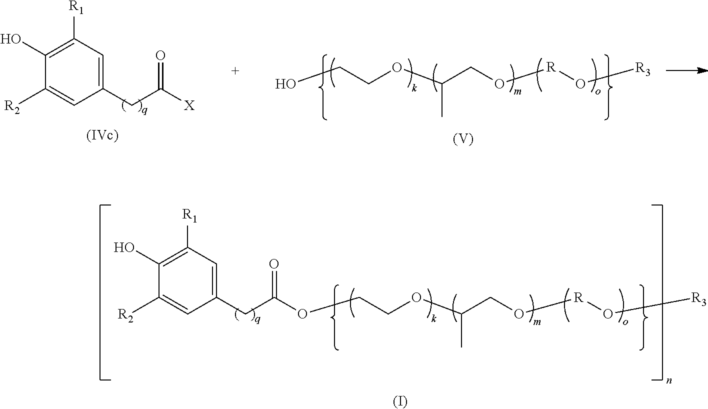

In the reaction schemes above, R, R₁, R₂, R₃ k, n, q, m and o have the definition given above for formula (I), R^{V} is a straight-chain or branched-chain alkyl group having 1 to 4 carbon atoms, such as a methyl, ethyl, propyl, isopropyl, butyl or isobutyl group, for example, preferably a methyl group, and X is a halogen atom such as a chlorine or bromine atom.

The starting materials of the formula (V) are adducts of alkylene oxides with various alcohols, and may be described as alkylene glycol alkyl monoethers or as O-alkylated alkylene glycols. It should be borne in mind that in the case of polyalkylene oxide adducts, the products available commercially are often mixtures of two or more individual compounds having different numbers of ethylene oxide, propylene oxide and/or R-oxide units. If a mixture of this kind is used as starting material, the end product of the formula (I) consists of a corresponding mixture. It is therefore implicit that in the case of such mixtures, the indices k, m and o may denote an average number of ethylene oxide, propylene oxide and R-oxide units, respectively, and so they may be fractional numbers for the overall mixture.

Process A encompasses a transesterification between the alkylene glycol monoether of the formula (V) and the substituted phenolpropionic ester of the formula (IVa). This reaction can be carried out as desired in the presence or absence of a solvent and in the presence of a transesterification catalyst.

If a solvent is used in this reaction, examples of suitable inert solvents include ethers such as diisopropyl ether, dioxane and tetrahydrofuran, halogenated hydrocarbons such as carbon tetrachloride and dichloroethane, linear or cyclic aliphatic hydrocarbons such as hexane, heptane, octane, isooctane, cyclohexane, methylcyclohexane, ethylcyclohexane and kerosine, and aromatic hydrocarbons such as benzene, toluene and xylene. Aromatic hydrocarbons are preferred.

Examples of suitable transesterification catalysts include alkali metals such as lithium, sodium and potassium, alkali metal hydrides such as lithium hydride, alkali metal amides such as lithium amide, sodium amide and lithium N,N-diisopropylamide, alkali metal alkoxides such as sodium methoxide, sodium ethoxide and potassium tert-butoxide, alkali metal hydroxides such as lithium hydroxide, sodium hydroxide and potassium hydroxide, alkali metal carbonates such as lithium carbonate, sodium carbonate and potassium carbonate, alkali metal bicarbonates such as lithium bicarbonate, sodium bicarbonate and potassium bicarbonate, carboxylic salts of alkali metals and alkaline earth metals (for example acetates or formates) such as lithium acetate, sodium acetate, potassium acetate or magnesium acetate and lithium formate, sodium formate or potassium formate, aluminum alkoxides and phenoxides, titanium(IV) alkoxides such as titanium(IV) tetraisopropoxide and titanium(IV) tetrabutoxide, metal oxides such as tin oxide, metal-organic tin(IV) compounds such as dibutyltin oxide, or organic acids such as benzenesulphonic acid, p-toluenesulphonic acid, trifluoromethanesulphonic acid and methanesulphonic acid, and mineral acids such as hydrochloric acid and sulphuric acid, preference being given to mineral acids and sulphonic acids, especially sulphuric acid as mineral acid and p-toluenesulphonic acid as sulphonic acid. The alkali metal alkoxides are preferred.

Reaction temperature and reaction time may vary depending on the starting materials, the catalyst and the solvent (where used). The temperature, however, is generally 50 to 200° C., more preferably 80 to 140° C., while the reaction time is commonly 2 to 24 hours, more preferably 4 to 12 hours.

After the ending of the transesterification reaction, the desired product of the formula (I) may be isolated by conventional techniques. For example, in the case of a basic reaction regime, the reaction mixture is washed and neutralized with a dilute mineral acid (e.g. dilute hydrochloric acid or sulphuric acid), after which insoluble constituents are removed (by filtration, for example) and the resulting liquid is dried over a dehydrating agent (e.g. anhydrous magnesium sulphate) and the solvent is evaporated. If desired, the product obtained may be purified, for example by distillation under reduced pressure or by column chromatography.

Process B encompasses the esterification of the alkylene glycol monoether of the formula (V) with the substituted phenol propionic acid of the formula (IVb). This reaction is preferably carried out in an inert solvent and in the presence of an acid catalyst.

Examples of suitable inert solvents which can be used in this reaction include ethers such as diisopropyl ether, dioxane and tetrahydrofuran, halogenated hydrocarbons such as methylene chloride, carbon tetrachloride and dichloroethane, aliphatic hydrocarbons such as hexane, heptane, octane, ethylcyclohexane and kerosine, and aromatic hydrocarbons such as benzene, toluene and xylene. Aromatic hydrocarbons are preferred.

The acid catalysts which can be used in this reaction include, for example, sulphonic acids such as benzenesulphonic acid, p-toluenesulphonic acid, trifluoromethanesulphonic acid and methanesulphonic acid, and mineral acids such as hydrochloric acid and sulphuric acid, preference being given to mineral acids and sulphonic acids, especially sulphuric acid as mineral acid and p-toluenesulphonic acid as sulphonic acid.

Reaction temperature and reaction time may vary depending on the starting materials, the solvent and the catalyst, although the temperature is generally 60 to 200° C., more preferably 100 to 150° C., and the reaction time is generally 3 to 24 hours, more preferably 4 to 12 hours.

After the ending of the esterification reaction, the desired product of the formula (I) may be isolated by conventional techniques. For example, the reaction mixture is washed and neutralized with an aqueous alkali metal solution (e.g. aqueous sodium bicarbonate), after which insoluble constituents are removed (by filtration, for example) and the liquid obtained is dried over a dehydrating agent (e.g. anhydrous magnesium sulphate) and the solvent is evaporated, giving the product of the formula (I). If desired, the product may be purified, for example by distillation under reduced pressure or by column chromatography.

Process C encompasses the esterification of the alkylene glycol monoether of the formula (V) with the substituted phenylpropionyl halide of the formula (IVc). This reaction is carried out preferably in an inert solvent and in the presence of a hydrogen halide scavenger.

Examples of solvents suitable in this reaction include those already listed for the reaction of process A.

Examples of suitable hydrogen halide scavengers include alkali metal hydroxides such as lithium hydroxide, sodium hydroxide and potassium hydroxide, alkali metal carbonates such as lithium carbonate, sodium carbonate and potassium carbonate, alkali metal bicarbonates such as lithium bicarbonate, sodium bicarbonate and potassium bicarbonate, aliphatic tertiary amines such as triethylamine, trioctylamine, N-methylmorpholine and N,N-dimethylpiperazine, and pyridines such as pyridine and N,N-dimethylaminopyridine. Triethylamine and the pyridines are preferred.

Reaction temperature and reaction time may vary depending on the starting materials, the solvent and hydrogen halide scavenger that are used. However, the reaction temperature is commonly 0 to 120° C., more preferably 10 to 60° C., while the reaction time is commonly 1 hour to 12 hours, more preferably 4 to 8 hours.

After the ending of the reaction, the desired product of the formula (I) may be isolated by conventional techniques. For example, the reaction mixture is washed with a dilute mineral acid (e.g. dilute hydrochloric acid or sulphuric acid), after which insoluble constituents are removed (by filtration, for example) and the liquid obtained is dried over a dehydrating agent (e.g. anhydrous magnesium sulphate) and the solvent is evaporated, giving the desired product. If desired, the product can be purified, for example by distillation under reduced pressure or by column chromatography.

The compounds of the formula (I) of the invention also allow the provision of antioxidant mixtures. An antioxidant mixture in the sense of this invention comprises at least one further antioxidant as well as the compound of the formula (I). In addition, if desired, there may also be further ingredients, such as solvents etc., for example. These solvents are preferably selected from water, alcohols, especially polyether monools or polyols, preferably consisting of H-functional starter substances, onto which alkylene oxides (epoxides) having 2-24 carbon atoms, preferably ethylene oxide and/or propylene oxide, have been added by alkoxylation, and which have a molecular weight of preferably 200-8000 g/mol, more preferably of 300-5000 g/mol, very preferably of 500-1000 g/mol, and a PO content of preferably 10-100 wt %, more preferably of 50-100 wt %, and also polyester monools or polyols having a molecular weight preferably in the range from 200 to 4500 g/mol, glycols, alkoxylates, carbonates, ethers, esters, branched or linear aliphatic or aromatic hydrocarbons and/or oils of synthetic and/or natural origin.

The “further antioxidant” may be any known natural or synthetic antioxidant, more particularly those which are commonly used in connection with preventing the oxidative degradation of plastics, PU systems and/or adhesives.

Surprisingly, however, in the context of this invention, it has been found that specifically certain benzofuranone derivatives of the formula (II) cited below provide very particularly strong support for the efficacy of the compounds of the formula (I), allowing the term “synergistic interaction” to be used.

Compounds of the benzofuran-2-one type that are preferred accordingly in the sense of this invention are compounds of the formula (II).

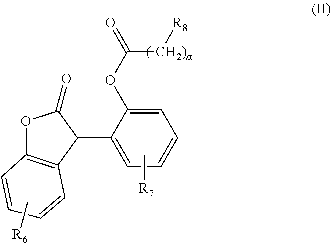

in which
a is an integer between 0 and 7, preferably 0-3, e.g. 1 or 2,
R₆ and R₇ independently of one another are H or C₁-C₈ alkyl, e.g. tert-butyl,

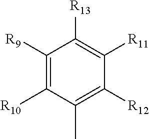

R₈ is H or an aromatic radical where
R₉ and R₁₀ independently of one another are H or C₁-C₆ alkyl, with not both being a C₁-C₆ alkyl,
R₁₁ and R₁₂ independently of one another are H or C₁-C₆ alkyl, with not both being a C₁-C₆ alkyl,
R₁₃ is H or OH, especially OH.

Benzofuran-2-one stabilizers of the formula (II) are known in the literature as such. Reference may be made here in particular to EP 2500341.

More particularly it is possible in the sense of this invention, as compound of the benzofuran-2-one type, to use the compound (IIa),
4-tert-butyl-2-(5-tert-butyl-2-oxo-2, 3-dihydro-1-benzofuran-3-yl)phenyl 3,5-di-tert-butyl-4-hydroxybenzoate (IIa).

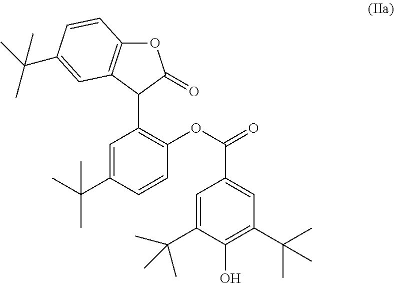 [SMILES: CC(C)(C)c1ccc(OC(=O)c2cc(C(C)(C)C)c(O)c(C(C)(C)C)c2)c(C2C(=O)Oc3ccc(C(C)(C)C)cc32)c1]

The use of this compound of the formula (IIa) has shown particularly advantageous results in respect of the desired effects.

According to one preferred embodiment of the invention, an antioxidant mixture of the invention comprises compound(s) of the formula (I) in an amount of 75 to 99 wt %, preferably 80 to 98 wt %, more particularly 90 to 95 wt %, and compound(s) of the formula (II) in an amount of 1 to 25 wt %, preferably 2 to 20 wt %, more particularly 5 to 10 wt %, wt % being based on the total weight of the compounds of the formulae (I) and (II) used.

It has additionally been found that antioxidant mixtures of the invention which comprise compound(s) of both formulae (I) and (II), particularly in combination with a phosphite (ester of phosphorous acid), are particularly advantageous. Phosphites which can be used with preference accordingly are those having the general formula (III)

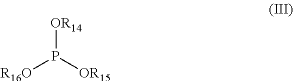

in which
R₁₄, R₁₅ and R₁₆ independently of one another are an aromatic or aliphatic, linear or branched radical of C₁-C₃₀-alkyl or C₂-C₃₀-alkylene, interrupted in each case optionally by one or more oxygen atoms.

Particularly preferred examples of such phosphites are the commercially available compounds (IIIa), (IIIb) and (IIIc).

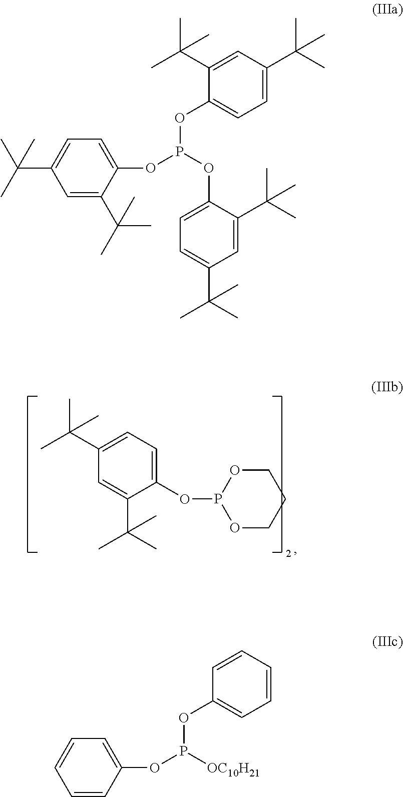

A compound of the phosphite type which can be used in particular in the sense of this invention is the compound (IIIa), tris(2,4-di-tert-butylphenyl) phosphite (IIIa).

The use of this compound of the formula (IIIa) has shown especially advantageous results in respect of the desired effects.

According to one especially preferred embodiment of the invention, an antioxidant mixture of the invention comprises compound(s) of the formula (I) in an amount of 75 to 99 wt %, preferably 80 to 98 wt %, more particularly 90 to 95 wt %, and compound(s) of the formula (II) in an amount of 1 to 25 wt %, preferably 2 to 20 wt %, more particularly 5 to 10 wt %, and compounds of the formula (III) in an amount of 0.1 to 20 wt %, preferably 0.2 to 15 wt %, more particularly 0.5 to 10 wt %, wt % being based on the total weight of the compounds of the formulae (I), (II) and (III) used.

A further subject of this invention against the outlined background is a process for producing polyurethane systems, especially PU foam, by reaction of at least one polyol component with at least one isocyanate component in the presence of one or more catalysts which catalyze the isocyanate-polyol and/or isocyanate-water reactions and/or the isocyanate trimerization, the reaction being carried out in the presence of one or more compounds of the formula (I) or in the presence of an antioxidant mixture, as described above.

The PU system, more particularly the PU foam, is preferably produced by foaming a mixture comprising at least one urethane catalyst and/or isocyanurate catalyst, at least one blowing agent and/or water, at least one isocyanate component and a polyol component in the presence of one or more compounds of the formula (I) or in the presence of an antioxidant mixture, as described above.

In addition to the stated components, the mixture may have other constituents, such as, for example, optionally (further) blowing agents, optionally prepolymers, optionally flame retardants and optionally further additives (different from those identified in the additive composition of the invention), such as fillers, emulsifiers based on the reaction of hydroxy-functional compounds with isocyanates, foam stabilizers, such as Si-containing and non-Si-containing stabilizers, especially Si-containing and non-Si-containing organic stabilizers and surfactants, viscosity reducers, dyes, UV stabilizers or antistats, for example. It will be readily understood that the skilled person selects the substances necessary for producing the individual types of flexible polyurethane foam, i.e. hot-cure, cold-cure or ester foams of flexible polyurethane type, examples of such substances being isocyanate, polyol, prepolymer, etc., as appropriate for obtaining the particular desired type of flexible polyurethane foam.

A number of property rights describing suitable components and processes for producing the different types of flexible polyurethane foam, i.e. hot-cure, cold-cure and also ester flexible polyurethane foams, are indicated hereinbelow and are fully incorporated herein by reference: EP 0152878 A1, EP 0 409 035 A2, DE 102005050473 A1, DE 19629161 A1, DE 3508292 A1, DE 4444898 A1, EP 1061095 A1, EP 0532939 B1, EP 0867464 B1, EP 1683831 A1 and DE 102007046860 A1.

Further details of usable starting materials, catalysts and auxiliaries and additives can be found, for example, in Kunststoff-Handbuch [Plastics Handbook], volume 7, Polyurethane [Polyurethanes], Carl-Hanser-Verlag Munich, 1st edition 1966, 2nd edition 1983 and 3rd edition 1993.

The compounds, components and additives which follow are mentioned merely by way of example and can be replaced by other substances known to those skilled in the art.

Surfactants employable in the production of flexible polyurethane foams are selectable, for example, from the group comprising nonionic surfactants and/or amphoteric surfactants.

Surfactants used may, in accordance with the invention, for example, also be polymeric emulsifiers such as polyalkyl polyoxyalkyl polyacrylates, polyvinylpyrrolidones or polyvinyl acetates. It is likewise possible to use, as surfactants/emulsifiers, prepolymers which are obtained by reaction of small amounts of isocyanates with polyols (called oligourethanes), and which are preferably present dissolved in polyols.

Foam stabilizers used may preferably be those which are known from the prior art and which are typically also employed for polyurethane foam stabilization. These may be both Si-containing and non-Si-containing, especially Si-containing and non-Si-containing organic stabilizers and surfactants. The Si-containing stabilizers are further distinguished by whether the polyoxyalkylene block is bonded to the polysiloxane block by a hydrolytically stable C—Si bond (as, for example, in EP 2 182 020) or by the less hydrolytically stable C—O—Si bond. The SiC-polysiloxane-polyoxyalkylene block copolymers usable for polyurethane foam stabilization can be prepared, for example, by noble metal-catalyzed hydrosilylation of unsaturated polyoxyalkylenes with SiH-functional siloxanes, called hydrosiloxanes, as described, for example, in EP 1 520 870. The hydrosilylation can be conducted batchwise or continuously, as described, for example, in DE 198 59 759 C1.

A host of further specifications, such as EP 0493836 A1, U.S. Pat. No. 5,565,194 or EP 1350804, for example, each disclose polysiloxane-polyoxyalkylene block copolymers of specific composition for complying with specific profiles of requirements for foam stabilizers in diverse polyurethane foam formulations.

Biocides used may be commercial products such as chlorophene, benzisothiazolinone, hexahydro-1,3,5-tris(hydroxyethyl-s-triazine), chloromethylisothiazolinone, methylisothiazolinone or 1,6-dihydroxy-2,5-dioxohexane, which are known by the trade names BIT 10, Nipacide BCP, Acticide MBS, Nipacide BK, Nipacide CI, Nipacide FC.

Suitable flame retardants for the purposes of this invention are any substances considered suitable therefore in the prior art. Examples of preferred flame retardants are liquid organophosphorus compounds such as halogen-free organophosphates, e.g. triethyl phosphate (TEP), halogenated phosphates, e.g. tris(1-chloro-2-propyl) phosphate (TCPP), tris(1,3-dichloro-2-propyl) phosphate (TDCPP) and tris(2-chloroethyl) phosphate (TCEP), and organic phosphonates, e.g. dimethyl methanephosphonate (DMMP), dimethyl propanephosphonate (DMPP), or solids such as ammonium polyphosphate (APP) and red phosphorus. Suitable flame retardants further include halogenated compounds, for example halogenated polyols, and also solids such as expandable graphite and melamine. All of these flame retardants and combinations thereof may be utilized advantageously in the sense of this invention, and include all commercially available flame retardants from the companies Great Lakes Solutions (Chemtura) (e.g.: DP-54™, Firemaster® BZ-54 HP, Firemaster® Firemaster® 550, Firemaster® 552, Firemaster® 600, Firemaster® 602, Reofos® 50, Reofos® 65, Reofos® 95, Kronitex® CDP), ICL Industrial Products (e.g.: FR-513, FR-1210, FR-1410, Fyrol™ FR-2, Fyrol™ 38, Fyrol™ HF-5, Fyrol™ A300 TB, Fyrol™ PCF, Fyrol™ PNX, Fyrol™ PNX-LE), Clariant (e.g.: Exolit® OP 550 or Exolit® OP 560).

In many cases, all of the components apart from the polyols and isocyanates are mixed prior to foaming, to give what is called an activator solution. This solution then preferably comprises, among other ingredients, the additive composition which can be used in accordance with the invention, i.e. compounds of the formula (I) or antioxidant mixture of the invention, foam stabilizers, catalysts or catalyst combination, the blowing agent, water for example, and any further additives, such as flame retardency, color, biocides, etc., depending on the formula for the flexible polyurethane foam. An activator solution of this type may also be a composition according to the invention.

With regard to the blowing agents, a distinction is made between chemical and physical blowing agents. The chemical blowing agents include, for example, water, the reaction of which with the isocyanate groups leads to formation of CO₂. The apparent density of the foam can be controlled by the amount of water added, with the preferred amounts of water used lying, for example, between 0.5 and 10 parts, preferably between 1 and 7 parts, more preferably between 1 and 5 parts, based on 100.0 parts of polyol. In addition, it is alternatively and/or else additionally possible to use physical blowing agents. These are liquids which are inert to the formulation constituents and have boiling points below 100° C., preferably below 50° C., especially between −50° C. and 30° C., at atmospheric pressure, such that they evaporate under the influence of the exothermic polyaddition reaction. Examples of such liquids usable with preference are ketones such as acetone and/or methyl ethyl ketone, hydrocarbons such as n-, iso- or cyclopentane, n- or isobutane and propane, cyclohexane, ethers such as dimethyl ether and diethyl ether, halogenated hydrocarbons such as methylene chloride, tetrafluoroethane, pentafluoropropane, heptafluoropropane, pentafluorobutane, hexafluorobutane and/or dichloromonofluoroethane, trichlorofluoromethane, dichlorotetrafluoroethane and 1,1,2-trichloro-1,2,2-trifluoroethane. In addition, it is also possible to use carbon dioxide. It is also possible to use mixtures of these low-boiling liquids with one another and/or with other substituted or unsubstituted hydrocarbons. The foaming may proceed either under standard pressure or under reduced pressure (VPF technology).

The amount of the physical blowing agent in this case is preferably in the range between 1 and 120 parts by weight, more particularly between 1 and 15 parts by weight, and the amount of water is preferably in the range between 0.5 to 10 parts by weight, more particularly 1 to 5 parts by weight, based in each case on 100 parts by weight of polyol. Carbon dioxide is preferred among the physical blowing agents, and is preferably used in combination with water as chemical blowing agent.

The inventive activator solution may additionally comprise all the customary additives known for activator solutions in the prior art. The additions may be selected from the group encompassing flame retardants, UV stabilizers, dyes, biocides, pigments, cell openers, crosslinkers and the like.

For the production of a PU foam of the invention, more particularly a flexible polyurethane foam, a preferred procedure involves reacting a mixture (mix) of polyol, di- or polyfunctional isocyanate, inventive additive, i.e. compounds of the formula (I) or antioxidant mixture of the invention, amine catalyst, organopotassium—zinc and/or—tin compound or other metal-containing catalysts, foam stabilizer, blowing agent, preferably water to form CO₂ and, if necessary, addition of physical blowing agents, optionally with flame retardants, UV stabilizers, color pastes, biocides, fillers, crosslinkers or other customary processing aids being added. Such a mixture likewise constitutes a subject of the invention. A mixture comprising the additive for inventive use, i.e. compounds of the formula (I) or antioxidant mixture of the invention, and polyol likewise constitutes a subject of the invention.

Isocyanates used may be organic isocyanate compounds containing at least two isocyanate groups. In general, useful isocyanates are the aliphatic, cycloaliphatic, araliphatic and preferably aromatic polyfunctional isocyanates known per se. Isocyanates are more preferably used at from 60 to 140 mol %, relative to the sum total of isocyanate-consuming components.

Specific examples include the following: alkylene diisocyanates having 4 to 12 carbon atoms in the alkylene radical, such as dodecane 1,12-diisocyanate, 2-ethyltetramethylene 1,4-diisocyanate, 2-methylpentamethylene 1,5-diisocyanate, tetramethylene 1,4-diisocyanate and preferably hexamethylene 1,6-diisocyanate, cycloaliphatic diisocyanates such as cyclohexane 1,3- and 1,4-diisocyanates and any desired mixtures of these isomers, 1-isocyanato-3,3,5-trimethyl-5-isocyanatomethylcyclohexane (IPDI), hexahydrotolylene 2,4- and 2,6-diisocyanate and the corresponding isomer mixtures, dicyclohexylmethane 4,4′-, 2,2′- and 2,4′-diisocyanate and the corresponding isomer mixtures, and preferably aromatic di- and polyisocyanates, for example tolylene 2,4- and 2,6-diisocyanate and the corresponding isomer mixtures, diphenylmethane 4,4′-, 2,4′- and 2,2′-diisocyanate and the corresponding isomer mixtures, mixtures of diphenylmethane 4,4′- and 2,2′-diisocyanates, polyphenylpolymethylene polyisocyanates, mixtures of diphenylmethane 4,4′-, 2,4′- and 2,2′-diisocyanates and polyphenylpolymethylene polyisocyanates (crude MDI) and mixtures of crude MDI and tolylene diisocyanates. Organic di- and polyisocyanates can be used individually or as mixtures thereof.

It is also possible to use isocyanates which have been modified by the incorporation of urethane, uretdione, isocyanurate, allophanate and other groups, called modified isocyanates.

Organic polyisocyanates have been found to be particularly useful and are therefore employed with preference:

tolylene diisocyanate, mixtures of diphenylmethane diisocyanate isomers, mixtures of diphenylmethane diisocyanate and polyphenylpolymethyl polyisocyanate or tolylene diisocyanate with diphenylmethane diisocyanate and/or polyphenylpolymethyl polyisocyanate or what are called prepolymers.

It is possible to use either TDI (tolylene 2,4- and 2,6-diisocyanate isomer mixture) or MDI (diphenylmethane 4,4′-diisocyanate). What is called “crude MDI” or “polymeric MDI” contains, as well as the 4,4′ isomers, also the 2,4′ and 2,2′ isomers, and also higher polycyclic products. “Pure MDI” refers to bicyclic products composed predominantly of 2,4′ and 4,4′ isomer mixtures or prepolymers thereof. Further suitable isocyanates are detailed in patent specification EP 1095968, to which reference is made here in full.

Crosslinkers refer to low molecular weight polyfunctional compounds that are reactive toward isocyanates. Suitable examples are polyfunctional, especially di- and trifunctional compounds having molecular weights of 62 to 1000 g/mol, preferably 62 to 600 g/mol. Those used include, for example, di- and trialkanolamines such as diethanolamine and triethanolamine, aliphatic and aromatic diamines, for example ethylenediamine, butylenediamine, butylene-1,4-diamine, hexamethylene-1,6-diamine, 4,4′-diaminodiphenylmethane, 3,3′-dialkyl-substituted 4,4′-diaminodiphenylmethanes, tolylene-2,4- and -2,6-diamine, and preferably aliphatic diols and triols having 2 to 6 carbon atoms, such as ethylene glycol, propylene glycol, 1,4-butylene glycol, 1,6-hexamethylene glycol, 2-methylpropane-1,3-diol, glycerol and trimethylolpropane or castor oil or pentaerythritol, and also higher polyhydric alcohols such as sugar alcohols, for example sucrose, glucose or sorbitol, and alkoxylated compounds of all the aforementioned examples.

The use concentration is typically between 0.1 and 5 parts, based on 100.0 parts polyol, according to the formulation, but may also differ therefrom. When MDI with a functionality f>2 is used in molded foaming, it likewise takes on a crosslinking function. Accordingly, with increasing amount of corresponding MDI, the amount of low molecular weight crosslinkers can be reduced.

The compositions according to the invention can be used in slabstock foaming. It is possible to use all processes known to those skilled in the art for production of free-rise flexible polyurethane foams. For example, the foaming operation can be effected either in the horizontal or in the vertical direction, in batchwise or continuous systems. The additive compositions usable in accordance with the present invention are similarly useful for CO₂ technology. Use in low-pressure and high-pressure machines is possible, in which case the formulations of the invention can be metered directly into the mixing chamber or else are added upstream of the mixing chamber to one of the components which subsequently pass into the mixing chamber. The addition can also be effected in the raw material tank.

Polyols suitable as polyol component for the purposes of the present invention are all organic substances having two or more isocyanate-reactive groups, preferably OH groups, and also formulations thereof. All polyether polyols and polyester polyols typically used for production of polyurethane systems, especially polyurethane foams, are preferred polyols.

These may, for example, be polyether polyols or polyester polyols which typically bear 2 to 8 OH groups per molecule and, as well as carbon, hydrogen and oxygen, may also contain heteroatoms such as nitrogen, phosphorus or halogens; preference is given to using polyether polyols. Polyols of this kind can be prepared by known processes, for example by anionic polymerization of alkylene oxides in the presence of alkali metal hydroxides or alkali metal alkoxides as catalysts, and with addition of at least one starter molecule containing preferably 2 to 3 reactive hydrogen atoms in bound form, or by cationic polymerization of alkylene oxides in the presence of Lewis acids, for example antimony pentachloride or boron fluoride etherate, or by double metal cyanide catalysis. Suitable alkylene oxides contain from 2 to 4 carbon atoms in the alkylene moiety. Examples are tetrahydrofuran, 1,2-propylene oxide, 1,2- or 2,3-butylene oxide; preference is given to using ethylene oxide and/or 1,2-propylene oxide. The alkylene oxides may be used individually, in alternation or as mixtures. H-functional starter substances used are especially polyfunctional alcohols and/or amines. Alcohols used with preference are dihydric alcohols, for example ethylene glycol, propylene glycol, or butanediols, trihydric alcohols, for example glycerol, trimethylolpropane or castor oil or pentaerythritol, and higher polyhydric alcohols, such as sugar alcohols, for example sucrose, glucose or sorbitol Amines used with preference are aliphatic amines having up to 10 carbon atoms, for example ethylenediamine, diethylenetriamine, propylenediamine, aromatic amities, for example tolylenediamine or diaminodiphenylmethane, and also amino alcohols such as ethanolamine or diethanolamine.

Polyester polyols can be prepared by a polycondensation reaction or by ring-opening polymerization. Acid components used are, for example, succinic acid, maleic acid, maleic anhydride, adipic acid, phthalic anhydride, phthalic acid, isophthalic acid, terephthalic acid, tetrahydrophthalic acid, tetrahydrophthalic anhydride, hexahydrophthalic anhydride or mixtures of said acids and/or anhydrides. Alcohol components used are, for example, ethanediol, 1,2-propanediol, 1,3-propanediol, 1,4-butanediol, 1,5-pentanediol, neopentyl glycol, 1,6-hexanediol, 1,4-bis(hydroxymethyl)cyclohexane, diethylene glycol, dipropylene glycol, trimethylolpropane, glycerol, pentaerythritol or mixtures of the stated alcohols. If the alcohol component used is dihydric or polyhydric polyether polyols, the result is polyester ether polyols which can likewise serve as starter substances for preparation of the polyether polycarbonate polyols. Preference is given to using polyether polyols having Mn=150 to 2000 g/mol for preparation of the polyester ether polyols.

The polyether polyols, preferably polyoxypropylenepolyoxyethylene polyols, typically have a functionality of 2 to 8 and number-averaged molecular weights preferably in the range from 500 to 8000, preferably 800 to 4500. Further polyols are known to those skilled in the art and can be found, for example, in EP-A-0380993 or U.S. Pat. No. 3,346,557, to which reference is made in full.

High-elasticity flexible polyurethane foams (cold-cure foam) are preferably produced by employing di- and/or trifunctional polyether alcohols preferably having above 50 mol % of primary hydroxyl groups, based on the sum total of hydroxyl groups, in particular those having an ethylene oxide block at the chain end or those based exclusively on ethylene oxide.

Slabstock flexible foams are preferably produced by employing di- and/or tri-functional polyether alcohols having secondary hydroxyl groups, preferably above 80 mol %, in particular those having a propylene oxide block or random propylene oxide and ethylene oxide block at the chain end, or those based exclusively on propylene oxide blocks.

A further class of polyols is of those which are obtained as prepolymers by reaction of polyol with isocyanate in a molar ratio of 100:1 to 5:1, preferably 50:1 to 10:1. Such prepolymers are preferably used in the form of a solution in polyol, and the polyol preferably corresponds to the polyol used for preparing the prepolymers.

Yet a further class of polyols is that of the so-called filled polyols (polymer polyols). These contain dispersed solid organic fillers up to a solids content of 40% by weight or more. Those used include the following:

SAN polyols: These are highly reactive polyols containing a dispersed copolymer based on styrene-acrylonitrile (SAN).

PHD polyols: These are highly reactive polyols containing polyurea, likewise in dispersed form.

PIPA polyols: These are highly reactive polyols containing a dispersed polyurethane, for example formed by in situ reaction of an isocyanate with an alkanolamine in a conventional polyol.

The solids content, which is preferably between 5% and 40% by weight, based on the polyol, depending on the application, is responsible for improved cell opening, and so the polyol can be foamed in a controlled fashion, especially with TDI, and no shrinkage of the foams occurs. The solids content thus acts as an essential processing aid. A further function is to control the hardness via the solids content, since higher solids contents bring about a higher hardness on the part of the foam.

The formulations with solids-containing polyols have distinctly lower intrinsic stability and therefore tend also to additionally require physical stabilization in addition to the chemical stabilization due to the crosslinking reaction.

Depending on the solids contents of the polyols, these are used alone or in a blend with the abovementioned unfilled polyols.

A further class of useful polyols is that of the so-called autocatalytic polyols, in particular autocatalytic polyether polyols. Polyols of this kind are based, for example, on polyether blocks, preferably on ethylene oxide and/or propylene oxide blocks, and additionally include catalytically active functional groups, for example nitrogen-containing functional groups, especially amino groups, preferably tertiary amine functions, urea groups and/or heterocycles containing nitrogen atoms. Through the use of such autocatalytic polyols in the production of polyurethane systems, especially of polyurethane foams, more preferably of flexible polyurethane foams, the requisite amount of any catalysts used may optionally be reduced, according to application, and/or adapted to specific desired foam properties. Suitable polyols are described, for example, in WO0158976 (A1), WO2005063841 (A1), WO0222702 (A1), WO2006055396 (A1), WO03029320 (A1), WO0158976 (A1), U.S. Pat. No. 6,924,321 (B2), U.S. Pat. No. 6,762,274 (B2), EP2104696 (B1), WO2004060956 (A1) or WO2013102053 (A1) and can be purchased, for example, under the Voractiv™ and/or SpecFlex™ Activ trade names from Dow.

Blowing agents used may be the known blowing agents. Preferably, in the production of the polyurethane foam, water, methylene chloride, pentane, alkanes, halogenated alkanes, acetone and/or carbon dioxide are used as blowing agents.

The water can be added directly to the mixture or else be added to the mixture as a secondary component of one of the reactants, for example of the polyol component, together with the latter.

In addition to physical blowing agents and any water, it is also possible to use other chemical blowing agents which react with isocyanates to evolve a gas, an example being formic acid.

Catalysts used in the context of this invention may, for example, be any catalysts for the isocyanate-polyol (urethane formation) and/or isocyanate-water (amine and carbon dioxide formation) and/or isocyanate dimerization (uretdione formation), isocyanate trimerization (isocyanurate formation), isocyanate-isocyanate with CO₂ elimination (carbodiimide formation) and/or isocyanate-amine (urea formation) reactions and/or “secondary” crosslinking reactions such as isocyanate-urethane (allophanate formation) and/or isocyanate-urea (biuret formation) and/or isocyanate-carbodiimide (uretonimine formation).

Suitable catalysts for the purposes of the present invention are, for example, substances which catalyse one of the aforementioned reactions, especially the gelling reaction (isocyanate-polyol), the blowing reaction (isocyanate-water) and/or the dimerization or trimerization of the isocyanate. Such catalysts are preferably nitrogen compounds, especially amines and ammonium salts, and/or metal compounds.

Suitable nitrogen compounds as catalysts, also referred to hereinafter as nitrogenous catalysts, for the purposes of the present invention are all nitrogen compounds according to the prior art which catalyse one of the abovementioned isocyanate reactions and/or can be used for production of polyurethanes, especially of polyurethane foams.

Examples of suitable nitrogen compounds as catalysts for the purposes of the present invention are preferably amines, especially tertiary amines or compounds containing one or more tertiary amine groups, including the amines triethylamine, N,N-dimethylcyclohexylamine, N,N-dicyclohexylmethylamine, N,N-dimethylaminoethylamine, N,N,N′,N′-tetramethylethylene-1,2-diamine, N,N,N′,N′-tetramethylpropylene-1,3-diamine, N,N,N′,N′-tetramethyl-1,4-butanediamine, N,N,N′,N′-tetramethyl-1,6-hexanediamine, N,N,N′,N″,N″-pentamethyldiethylenetriamine, N,N,N′-trimethylaminoethylethanolamine, N,N-dimethylaminopropylamine, N,N-diethylaminopropylamine, N,N-dimethylaminopropyl-N′,N′-dipropan-2-olamine, 2-[[3-(dimethylamino)propyl]methylamino]ethanol, 3-(2-dimethylamino)ethoxy)propylamine, N,N-bis[3-(dimethylamino)propyl]amine, N,N,N′,N″,N″-pentamethyldipropylenetriamine, 1-[bis[3-(dimethylamino)propyl]amino]-2-propanol, N,N-bis[3-(dimethylamino)propyl]-N′,N′-dimethylpropane-1,3-diamine, triethylenediamine, 1,4-diazabicyclo[2.2.2]octane-2-methanol, N,N′-dimethylpiperazine, 1,2-dimethylimidazole, N-(2-hydroxypropyl)imidazole, 1-isobutyl-2-methylimidazole, N-(3-aminopropyl)imidazole, N-methylimidazole, N-ethylmorpholine, N-methylmorpholine, 2,2,4-trimethyl-2-silamorpholine, N-ethyl-2,2-dimethyl-2-silamorpholine, N-(2-aminoethyl)morpholine, N-(2-hydroxyethyl)morpholine, 2,2′-dimorpholinodiethyl ether, N,N′-dimethylpiperazine, N-(2-hydroxyethyl)piperazine, N-(2-aminoethyl)piperazine, N,N-dimethylbenzylamine, N,N-dimethylaminoethanol, N,N-diethylaminoethanol, 3-dimethylamino-1-propanol, N,N-dimethylaminoethoxyethanol, N,N-diethylaminoethoxyethanol, bis(2-dimethylaminoethyl ether), N,N,N′-trimethyl-N′-(2-hydroxyethyl)bis(2-aminoethyl) ether, N,N,N-trimethyl-N-3′-aminopropyl(bisaminoethyl) ether, tris(dimethylaminopropyl)hexahydro-1,3,5-triazine, 1,8-diazabicyclo[5.4.0]undec-7-ene, 1,5-diazabicyclo[4.3.0]non-5-ene, 1,5,7-triazabicyclo[4.4.0]dec-5-ene, N-methyl-1,5,7-triazabicyclo[4.4.0]dec-5-ene, 1,4,6-triazabicyclo[3.3.0]oct-4-ene, 1,1,3,3-tetramethylguanidine, tert-butyl-1,1,3,3-tetramethylguanidine, guanidine, 3-dimethylaminopropylurea, 1,3-bis[3-(dimethylamino)propyl]urea, bis-N,N-(dimethylaminoethoxyethyl)isophoronedicarbamate, 3-dimethylamino-N,N-dimethylpropionamide and 2,4,6-tris(dimethylaminomethyl)phenol. Suitable nitrogenous catalysts according to the prior art can be purchased, for example, from Evonik under the TEGOAMIN® trade name.

According to the application, it may be preferable that, in the inventive production of polyurethane foams, quaternized and/or protonated nitrogenous catalysts, especially quaternized and/or protonated tertiary amines, are used.

For possible quaternization of nitrogenous catalysts, it is possible to use any reagents known as quaternizing reagents. Preferably, quaternizing agents used are alkylating agents, for example dimethyl sulphate, methyl chloride or benzyl chloride, preferably methylating agents such as dimethyl sulphate in particular. Quaternization is likewise possible with alkylene oxides, for example ethylene oxide, propylene oxide or butylene oxide, preferably with subsequent neutralization with inorganic or organic acids.

Nitrogenous catalysts, if quaternized, may be singly or multiply quaternized. Preferably, the nitrogenous catalysts are only singly quaternized. In the case of single quaternization, the nitrogenous catalysts are preferably quaternized on a tertiary nitrogen atom.

Nitrogenous catalysts can be converted to the corresponding protonated compounds by reaction with organic or inorganic acids. These protonated compounds may be preferable, for example, when, for example, a slowed polyurethane reaction is to be achieved or when the reaction mixture is to have enhanced flow in use.

Useful organic acids include, for example, any hereinbelow recited organic acids, for example carboxylic acids having 1 to 36 carbon atoms (aromatic or aliphatic, linear or branched), for example formic acid, lactic acid, 2-ethylhexanoic acid, salicylic acid and neodecanoic acid, or else polymeric acids such as, for example, polyacrylic or polymethacrylic acids. Inorganic acids used may, for example, be phosphorus-based acids, sulphur-based acids or boron-based acids.

However, the use of nitrogenous catalysts which have not been quaternized or protonated is particularly preferred in the context of this invention.

Suitable metal compounds as catalysts, also referred to hereinafter as metallic catalysts, for the purposes of the present invention are all metal compounds according to the prior art which catalyse one of the abovementioned isocyanate reactions and/or can be used for production of polyurethanes, especially of polyurethane foams. They may be selected, for example, from the group of the metal-organic or organometallic compounds, metal-organic or organometallic salts, organic metal salts, inorganic metal salts, and from the group of the charged or uncharged metallic coordination compounds, especially the metal chelate complexes.

The expression “metal-organic or organometallic compounds” in the context of this invention especially encompasses the use of metal compounds having a direct carbon-metal bond, also referred to here as metal organyls (e.g. tin organyls) or organometallic compounds (e.g. organotin compounds). The expression “organometallic or metal-organic salts” in the context of this invention especially encompasses the use of metal-organic or organometallic compounds having salt character, i.e. ionic compounds in which either the anion or cation is organometallic in nature (e.g. organotin oxides, organotin chlorides or organotin carboxylates). The expression “organic metal salts” in the context of this invention especially encompasses the use of metal compounds which do not have any direct carbon-metal bond and are simultaneously metal salts, in which either the anion or the cation is an organic compound (e.g. tin(II) carboxylates). The expression “inorganic metal salts” in the context of this invention especially encompasses the use of metal compounds or of metal salts in which neither the anion nor the cation is an organic compound, e.g. metal chlorides (e.g. tin(II) chloride), pure metal oxides (e.g. tin oxides) or mixed metal oxides, i.e. containing a plurality of metals, and/or metal silicates or aluminosilicates. The expression “coordination compound” in the context of this invention especially encompasses the use of metal compounds formed from one or more central particles and one or more ligands, the central particles being charged or uncharged metals (e.g. metal- or tin-amine complexes). The expression “metal-chelate complexes” is to be understood for the purposes of this invention as comprehending in particular the use of metal-containing coordination compounds wherein the ligands have at least two sites for coordinating or binding with the metal center (e.g. metal- or to be more precise tin-polyamine or metal- or to be more precise tin-polyether complexes).

Suitable metal-containing compounds, especially as defined above, as catalysts in the sense of the present invention may be selected, for example, from all metal-containing compounds comprising lithium, sodium, potassium, magnesium, calcium, scandium, yttrium, titanium, zirconium, vanadium, niobium, chromium, molybdenum, tungsten, manganese, cobalt, nickel, copper, zinc, mercury, aluminum, gallium, indium, germanium, tin, lead, and/or bismuth, especially sodium, potassium, magnesium, calcium, titanium, zirconium, molybdenum, tungsten, zinc, aluminum, tin and/or bismuth, more preferably tin, bismuth, zinc and/or potassium.

Suitable organometallic salts and organic metal salts, as defined above, as catalysts for the purposes of the present invention are, for example, organotin, tin, zinc, bismuth and potassium salts, in particular corresponding metal carboxylates, alkoxides, thiolates and mercaptoacetates, for example dibutyltin diacetate, dimethyltin dilaurate, dibutyltin dilaurate (DBTDL), dioctyltin dilaurate (DOTDL), dimethyltin dineodecanoate, dibutyltin dineodecanoate, dioctyltin dineodecanoate, dibutyltin dioleate, dibutyltin bis(n-lauryl mercaptide), dimethyltin bis(n-lauryl mercaptide), monomethyltin tris(2-ethylhexyl mercaptoacetate), dimethyltin bis(2-ethylhexyl mercaptoacetate), dibutyltin bis(2-ethylhexyl mercaptoacetate), dioctyltin bis(isooctyl mercaptoacetate), tin(II) acetate, tin(II) 2-ethylhexanoate (tin(II) octoate), tin(II) isononanoate (tin(II) 3,5,5-trimethylhexanoate), tin(II) neodecanoate, tin(II) ricinoleate, tin(II) oleate, zinc(II) acetate, zinc(II) 2-ethylhexanoate (zinc(II) octoate), zinc(II) isononanoate (zinc(II) 3,5,5-trimethylhexanoate), zinc(II) neodecanoate, zinc(II) ricinoleate, bismuth acetate, bismuth 2-ethylhexanoate, bismuth octoate, bismuth isononanoate, bismuth neodecanoate, potassium formate, potassium acetate, potassium 2-ethylhexanoate (potassium octoate), potassium isononanoate, potassium neodecanoate and/or potassium ricinoleate.

In the inventive production of polyurethane foams, it may be preferable to rule out the use of organometallic salts, for example of dibutyltin dilaurate.

Suitable metal-containing catalysts are generally selected with preference such that they do not have any inherent nuisance odor, are substantially unobjectionable toxicologically, and endow the resultant polyurethane systems, especially polyurethane foams, with as low a level of catalyst-induced emissions as possible.

In the inventive production of polyurethane foams, it may be preferable, according to the application, to use incorporable/reactive or high molecular weight catalysts. Catalysts of this kind may be selected, for example, from the group of the metal compounds, preferably from the group of the tin, zinc, bismuth and/or potassium compounds, especially from the group of the metal carboxylates of the aforementioned metals, for example the tin, zinc, bismuth and/or potassium salts of isononanoic acid, neodecanoic acid, ricinoleic acid and/or oleic acid, and/or from the group of the nitrogen compounds, especially from the group of the low-emission amines and/or the low-emission compounds containing one or more tertiary amine groups, for example described by the amines dimethylaminoethanol, N,N-dimethyl-N′,N′-di(2-hydroxypropyl)-1,3-diaminopropane, N,N-dimethylaminopropylamine, N,N,N′-trimethyl-N′-hydroxyethylbis(aminoethyl) ether, 6-dimethylaminoethyl-1-hexanol, N-(2-hydroxypropyl)imidazole, N-(3-aminopropyl)imidazole, aminopropyl-2-methylimidazole, N,N,N′-trimethylaminoethanolamine, 2-(2-(N,N-dimethylaminoethoxy)ethanol, N-(dimethyl-3-aminopropyl)urea derivatives and alkylaminooxamides, such as bis(N—(N′,N′-dimethylaminopropyl))oxamide, bis(N—(N′,N′-dimethylaminoethyl))oxamide, bis(N—(N′,N′-imidazolidinylpropyl)oxamide, bis(N—(N′,N′-diethylaminoethyl))oxamide, bis(N—(N′,N′-diethylaminopropyl)oxamide, bis(N—(N′,N′-diethylaminoethyl)oxamide, bis(N—(N′,N′-diethylimino-1-methylpropyl)oxamide, bis(N-(3-morpholinopropylyl)oxamide, and the reaction products thereof with alkylene oxides, preferably having a molar mass in the range between 160 and 500 g/mol, and compounds of the general formula:

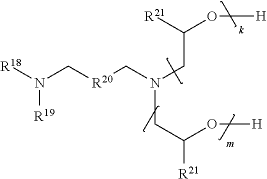

where
R18, R19=-CₐH₂ₐ₊ᵢ, where a=1-4 for acyclic groups
R18, R19=—C_{b}H_{c}N_{d} where b=3-7, c=6-14, d=0-2 for cyclic groups
R20=CₑH_{f}O₉ where e=0-4, f=0-8, g=0-2
R21=-H, —CH₃, —C₂H₅
k, m=identically or differently 1-5.

Catalysts and/or mixtures of this kind are supplied commercially, for example, under the Jeffcat® ZF-10, Lupragen® DMEA, Lupragen® API, Toyocat® RX 20 and Toyocat® RX 21, DABCO® RP 202, DABCO® RP 204, DABCO® NE 300, DABCO® NE 310, DABCO® NE 400, DABCO® NE 500, DABCO® NE 600, DABCO® NE 1060 and DABCO® NE 2039, Niax® EF 860, Niax® EF 890, Niax® EF 700, Niax® EF 705, Niax® EF 708, Niax® EF 600, Niax® EF 602, Kosmos® 54, Kosmos® EF and Tegoamin® ZE 1 name.

Suitable use amounts of catalysts are guided by the type of catalyst and are preferably in the range from 0.005 to 10.0 pphp, more preferably in the range from 0.01 to 5.00 pphp (=parts by weight based on 100 parts by weight of polyol) or 0.10 to 10.0 pphp for potassium salts.

According to the application, it may be preferable that, in the inventive production of polyurethane foams, one or more nitrogenous and/or metallic catalysts are used. When more than one catalyst is used, the catalysts may be used in any desired mixtures with one another. It is possible here to use the catalysts individually during the foaming operation, for example in the manner of a preliminary dosage in the mixing head, and/or in the form of a premixed catalyst combination.

The expression “premixed catalyst combination”, also referred to below as catalyst combination, encompasses, for the purposes of this invention, in particular, ready-made mixtures of metal-containing catalysts and/or nitrogen-containing catalysts and/or corresponding protonated and/or quaternized nitrogen-containing catalysts, and also, optionally, further ingredients or adjuvants such as, for example, water, organic solvents, acids to block the amines, emulsifiers, surfactants, blowing agents, antioxidants, flame retardants, foam stabilizers and/or siloxanes, preferably polyether siloxanes, which are already present as such prior to foaming and which do not need to be added as individual components during the foaming operation.

According to the application, it may be preferable when the sum total of all the nitrogenous catalysts used relative to the sum total of the metallic catalysts, especially potassium, zinc and/or tin catalysts, results in a molar ratio of 1:0.05 to 0.05:1, preferably 1:0.07 to 0.07:1 and more preferably 1:0.1 to 0.1:1.

In order to prevent any reaction of the components with one another, especially reaction of nitrogenous catalysts with metallic catalysts, especially potassium, zinc and/or tin catalysts, it may be preferable to store these components separately from one another and then to feed in the isocyanate and polyol reaction mixture simultaneously or successively.

By means of the process according to the invention, a polyurethane system, preferably polyurethane foam, especially a flexible polyurethane foam, is obtainable. This polyurethane system forms a further part of the subject-matter of the invention. The polyurethane foam in question is notable in particular for the fact that by virtue of the use of the inventive antioxidant additive the foam is a particularly low-emission foam.

In one preferred embodiment of the invention the polyurethane system comprises 0.0001 to 10 wt %, preferably 0.001 to 5 wt %, more particularly 0.01 to 3 wt %, based on the total weight of the polyurethane system, of one or more compounds of the formula (I) or of an antioxidant mixture, as described above.

With the polyurethane system, more particularly polyurethane foam, of the invention, articles are obtainable which comprise or consist of this polyurethane system, more particularly polyurethane foam. These articles represent a further subject of this invention. Articles of this kind may, for example, be furniture cushioning or mattresses.

A further subject of this invention, moreover, is a polyurethane system comprising the reaction products of one or more polyol components with one or more isocyanate components, where a hydroxyphenylcarboxylic ester of the formula (I)

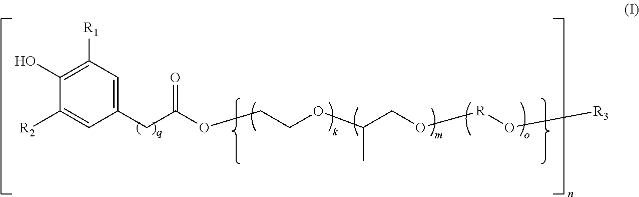

in which
R is CH₂—CH(R^{I}), CH(R^{II})—CH(R^{II}), CH₂—C(R^{II})₂, C(R^{II})₂—C(R^{II})₂,

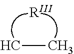

CH₂—CH—CH₂—R^{IV}, C₆H₆—CH—CH₂, or C₆H₆—C(CH₃)—CH₂, where
R^{I} is C₂ to C₂₄ alkyl radical or alkene radical, which may be linear or branched
R^{II} is C₂ to C₂₄ alkyl radical or alkene radical, which may be linear or branched
R^{III} is C₃ to C₆ alkyl radical, which is arranged linearly, and
R^{IV} is OH, Cl, OCH₃, OCH₂—CH₃, O—CH₂—CH═CH₂, O—CH═CH₂, molecule residue of singly or multiply epoxidized fats or oils as mono-, di-, and triglycerides, or molecule residue of singly or multiply epoxidized fatty acids or their C₁-C₂₄ alkyl esters,
R₁ and R₂ independently of one another are straight-chain or branched C₁-C₈ alkyl, cyclopentyl or cyclohexyl, especially tert-butyl,
q is 1, 2 or 3, preferably 2 or 3, especially 2,
n is an integer from 1 to 30, preferably an integer from 1 to 10, advantageously 1, 2, 3, 4, 5 or 6, e.g. 1, 2, 3 or 4, especially 1,
R₃ is an n-valent radical of linear or branched C₁-C₃₀ alkyl, preferably C₁-C₁₀ alkyl, C₂-C₃₀ alkylene, interrupted in each case optionally by one or more oxygen atoms, or (where n=1-12) is an n-valent radical of C₅-C₁₂ cycloalkyl, or a radical R₄—[NR₅—C_{q}H_{2q}—]ₚ,
R₄ is hydrogen, an n-valent radical of linear or branched C₁-C₃₀ alkyl, which is optionally interrupted by one or more groups —NR₅— or (where n=1-12) is an n-valent radical of C₅-C₁₂ cycloalkyl,
R₅ independently at each occurrence is hydrogen or methyl or —C_{q}H_{2q}—, preferably hydrogen, and
p corresponds to the number of —[NR₅—C_{q}H_{2q}-] groups that produces n radicals
   —C_{q}H_{2q}— per molecule,
k is an integer between 0 and 50, preferably between 10 and 30,
m is an integer between 0 and 50, e.g. 1-40, and
o is an integer between 0 and 50, preferably between 0 and 30, especially 0,
where (k+m+o)>10
   is employed as an aging inhibitor for synthetic polymers sensitive towards oxidative, thermal or light-induced degradation.

If in the formula (I) k, m, o>0 or k, m>0 and at the same time o=0, the sequence of the monomer units ethylene oxide, propylene oxide and (R-oxide) in the individual polymer chains 1 to n is arbitrary, and k, m and o represent average values. Moreover, the individual units (EO), (PO) and (RO) can be bonded to one another either in the form of blocks, in strict alternation or in the form of gradients.

A preferred composition of the invention for producing a polyurethane system, particularly polyurethane foam, may comprise polyol in amounts from 25 to 80 wt %, water in amounts from 1 to 5 wt %, catalysts in amounts from 0.05 to 1 wt %, physical blowing agent in amounts from 0 to 25 wt % (e.g. 0.1 to 25 wt %), foam stabilizers (such as, for example, Si-containing and non-Si-containing stabilizers, especially Si-containing and non-Si-containing organic stabilizers and surfactants) in amounts from 0.1 to 5 wt %, isocyanate in amounts from 20 to 50 wt %, and the additive for inventive use, i.e. compounds of the formula (I), or an antioxidant mixture of the invention, in amounts from 0.0001 to 10 wt %, preferably 0.001 to 5 wt %, more particularly 0.01 to 3 wt %, based on the total weight of the polyurethane system. The compound of the formula (I) is employed in particular in the form of an antioxidant mixture of the invention, comprising compounds of the formula (I) and also, preferably, compounds of the formula (II), more particularly comprising compounds of the formula (I) and also compounds of the formula (II) and (III).

For preferred embodiments of these abovementioned compositions, reference is made explicitly to the preceding description.

The invention further provides the use of the polyurethane systems obtainable in accordance with the invention as refrigerator insulation, insulant board, sandwich element, pipe insulation, sprayed foam, 1- and 1.5-component can foam, imitation wood, modelling foam, packaging foam, mattresses, furniture upholstery, material in motor vehicle interiors, vehicle seat upholstery, headrest, instrument panel, interior automotive trim, automotive roof liner, sound absorption material, steering wheel, footwear sole, carpet backing foam, filter foam, sealing foam, sealant and adhesive or for producing corresponding products, especially as material in motor vehicle interiors.

A further subject of the invention is the use of compounds of the formula (I) or antioxidant mixtures, as described above, for producing low-emission polyurethane systems, especially PU foam, with a reduced value for VOC and fogging. The compound of the formula (I) is employed in particular in the form of an antioxidant mixture of the invention, comprising compounds of the formula (I) and also, preferably, compounds of the formula (II), more particularly comprising compounds of the formula (I) and also compounds of the formula (II) and (III).

A further subject of the invention is the use of compounds of the formula (I) or antioxidant mixtures, as described above, for producing low-odor polyurethane systems, especially PU foam. The compound of the formula (I) is employed in particular in the form of an antioxidant mixture of the invention, comprising compounds of the formula (I) and also, preferably, compounds of the formula (II), more particularly comprising compounds of the formula (I) and also compounds of the formula (II) and (III).

A further subject of the invention is a method for lowering the total emission of organic compounds from polyurethane systems, especially polyurethane foams, by adding compounds of formula (I) to the polyurethane system, more particularly polyurethane foam, preferably in an amount of 0.0001 to 10 wt %, preferably 0.001 to 5 wt %, more particularly 0.01 to 3 wt %, based on the total weight of the polyurethane system, it being possible for the addition to be made before, during or after the production of the polyurethane system. The compound of the formula (I) is employed in particular in the form of an antioxidant mixture of the invention, comprising compounds of the formula (I) and also, preferably, compounds of the formula (II), more particularly comprising compounds of the formula (I) and also compounds of the formula (II) and (III).

The subject-matter of the present invention is elucidated in detail hereinafter with reference to examples, without any intention that the subject-matter of the invention be restricted to these illustrative embodiments.

### EXAMPLES

### Preparation of the Inventive Additive of the Formula (I)

The hydroxyphenylcarboxylic esters were prepared by process A as described above, starting from methyl 3-(3,5-di-tert-butyl-4-hydroxyphenyl)propionate (IVa-1).

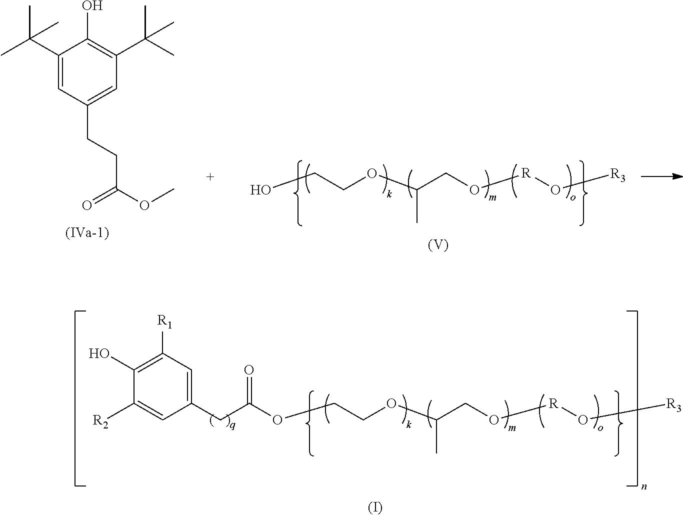

### Example 1 (Inventive)

A 250 mL three-necked flask with distillation bridge and KPG stirrer was charged with 29.3 g of the hydroxyphenylcarboxylic ester methyl 3-(3,5-di-tert-butyl-4-hydroxyphenyl)propionate (IVa-1) together with 61.9 g of a polyether of the general formula CH₃[CH₂CH₂O]₁₁H and 0.06 g of sodium acetate. The flask was flooded with nitrogen and the mixture was heated to 180° C. and stirred for an hour. The flask was then evacuated and the methanol formed was distilled off directly from the reaction mixture while stirring under reduced pressure (10 mbar) using a distillation bridge. After cooling had taken place, 6.9 g of the benzofuran-2-one (IIa) and 1.7 g of the phosphite (IIIa) were dissolved in the resulting liquid hydroxyphenylcarboxylic ester.

### Example 2 (Inventive)

A 250 mL three-necked flask with distillation bridge and KPG stirrer was charged with 29.3 g of the hydroxyphenylcarboxylic ester methyl 3-(3,5-di-tert-butyl-4-hydroxyphenyl)propionate (IVa-1) together with 75.8 g of a polyether of the general formula CH₃[CH₂CH(CH₃)O]₂[CH₂CH₂O]₁₁H and 0.55 g of titanium tetrabutoxide. The flask was flooded with nitrogen and the mixture was heated to 180° C. and stirred for an hour. The flask was then evacuated and the methanol formed was distilled off directly from the reaction mixture while stirring under reduced pressure (10 mbar) using a distillation bridge. After cooling had taken place, 7.9 g of the benzofuran-2-one (IIa) and 2.0 g of the phosphite (IIIa) were dissolved in the resulting liquid hydroxyphenylcarboxylic ester.

### Example 3 (Not Inventive)

A 250 ml three-necked flask with distillation bridge and KPG stirrer was charged with 29.3 g of the hydroxyphenylcarboxylic ester methyl 3-(3,5-di-tert-butyl-4-hydroxyphenyl)propionate (IVa-1) together with 15.6 g of 1-octanol and 0.2 g of para-toluenesulphonic acid. The flask was flooded with nitrogen and the mixture was heated to 180° C. and stirred for an hour. The flask was then evacuated and the methanol formed was distilled off directly from the reaction mixture while stirring under reduced pressure (10 mbar) using a distillation bridge. After cooling had taken place, 3.5 g of the benzofuran-2-one (IIa) and 0.9 g of the phosphite (IIIa) were dissolved in the resulting liquid hydroxyphenylcarboxylic ester.

### Production of the Polyurethane Foams

For the performance tests, three typical formulations of flexible polyurethane foams were used, with the following compositions:

TABLE 1
Formulation I for TDI80 flexible slabstock foam
applications for testing of the anti-scorch performance
(scorch = degradation of the polyurethane)

| Formulation I | Parts by mass (pphp) |
|---|---|
| Voranol ® CP 3322¹⁾ | 100 |
| Desmodur ® T 80²⁾ Index <104> | 56.4 |
| Water | 4.8 |
| TEGOAMIN ® 33³⁾ | 0.2 |
| KOSMOS ® 29⁴⁾ | 0.22 |
| Fyrol ™ A300TB⁵⁾ | 10.0 |
| TEGOSTAB ® B 8242⁶⁾ | 1.0 |
| Antioxidant mixture⁷⁾ | 1.0 |

¹⁾available from Dow Chemical; this is a glycerol-based polyether polyol having an OH number of 48 mg of KOH/g.
²⁾tolylene diisocyanate T 80 (80% 2,4 isomer, 20% 2,6 isomer) from Bayer MaterialScience, 3 mPa · s, 48% NCO, functionality 2.
³⁾amine catalyst from Evonik Industries AG.
⁴⁾tin catalyst, available from Evonik Industries AG.
⁵⁾phosphorus-based flame retardant from ICL Industrial Products.
⁶⁾polyether-modified polysiloxane, available from Evonik Industries AG.
⁷⁾inventive antioxidant mixture, prepared according to Examples 1-2, non-inventive antioxidant mixture, prepared according to Example 3, or antioxidant mixture ORTEGOL ® AO 5 from Evonik Industries AG.

TABLE 2
Formulation II for TDI80 flexible slabstock foam
applications for determining the VOC and fog emissions
as per DaimlerChrysler testing instructions VDA 278

| Formulation II | Parts by mass (pphp) |
|---|---|
| Arcol ® 1105 S⁸⁾ | 100 |
| Desmodur ® T 80²⁾ Index <110> | 41.6 |
| Water | 3.0 |
| TEGOAMIN ® ZE1³⁾ | 0.15 |
| KOSMOS ® EF⁴⁾ | 0.6 |
| TEGOSTAB ® BF 2370⁶⁾ | 0.8 |
| Antioxidant mixture⁷⁾ | 1.0 |

²⁾tolylene diisocyanate T 80 (80% 2,4 isomer, 20% 2,6 isomer) from Bayer MaterialScience, 3 mPa · s, 48% NCO, functionality 2.
³⁾amine catalyst from Evonik Industries AG.
⁴⁾tin catalyst, available from Evonik Industries AG
⁶⁾polyether-modified polysiloxane, available from Evonik Industries AG.
⁷⁾inventive antioxidant mixture, prepared according to Examples 1-2, non-inventive antioxidant mixture, prepared according to Example 3, or antioxidant mixture ORTEGOL ® AO 5 from Evonik Industries AG.
⁸⁾available from Bayer Material Science; this is a glycerol-based polyether polyol having an OH number of 56 mg of KOH/g.

TABLE 3
Formulation III for TDI80 flexible slabstock foam
applications for determining the VOC and fog emissions
as per DaimlerChrysler testing instructions VDA 278

| Formulation III | Parts by mass (pphp) |
|---|---|
| Arcol ® 1105 S⁸⁾ | 100 |
| Desmodur ® T 80²⁾ Index <110> | 62.9 |
| Water | 5.0 |
| TEGOAMIN ® ZE1³⁾ | 0.15 |
| KOSMOS ® EF⁴⁾ | 0.6 |
| TEGOSTAB ® BF 2370⁶⁾ | 1.0 |
| Antioxidant mixture⁷⁾ | 1.0 |

²⁾tolylene diisocyanate T 80 (80% 2,4-isomer, 20% 2,6-isomer) from Bayer MaterialScience, 3 mPa · s, 48% NCO, functionality 2.
³⁾amine catalyst from Evonik Industries AG.
⁴⁾tin catalyst, available from Evonik Industries AG
⁶⁾polyether-modified polysiloxane, available from Evonik Industries AG.
⁷⁾inventive antioxidant mixture, prepared according to Examples 1-2, non-inventive antioxidant mixture, prepared according to Example 3, or antioxidant mixture ORTEGOL ® AO 5 from Evonik Industries AG.
⁸⁾available from Bayer Material Science; this is a glycerol-based polyether polyol having an OH number of 56 mg of KOH/g.

### General Procedure for Production of the Foams

The foams were produced at 22° C. and air pressure 753 mmHg according to the details which follow. For the production of the polyurethane foams for the microwave test, 100 g of polyol were used in each case; for the production of the polyurethane foam for the oven test and the odor testing, 300 g of polyol were used in each case; for the production of the polyurethane foams for determining the VOC and fog emissions, 500 g of polyol were used in each case; the other formulation constituents were converted accordingly.

For example, 1.0 part (1.0 pphp) of a component here denoted 1 g of this substance per 100 g of polyol.

For the foaming, the polyol, water, catalyst (amine(s) and/or the tin compound), stabilizer and the antioxidant mixture used were mixed thoroughly with stirring. Following the addition of the isocyanate, stirring took place with a stirrer at 3000·rpm for 7 seconds and the mixture was poured into a paper-lined wooden box. Resultant flexible polyurethane foams were subjected to the performance tests described below.

For the demonstration of the anti-scorch performance of the present invention, a formulation was selected which is water-blown and freely risen (foam is able to rise unhindered; not molded foams). The amount of water was chosen as 4.8 parts per 100 parts polyol mixture. On the basis of this amount of water, a density of about 21 kg/m³ can be expected. In terms of density and amount of water, therefore, the formulation is typical of flexible polyurethane foam grades which are currently in use in the industry.

For the determination of the VOC and fog emissions, two different formulations were selected, but both were water-blown and freely risen (foam is able to rise unhindered; not molded foams). In the first case, the amount of water was chosen as 3 parts per 100 parts polyol; in the second case, 5 parts of water were used per 100 parts polyol. In this way, foams having densities of approximately 30 kg/m³ and 17 kg/m³ were obtained, respectively. As a result of the different amounts of water in the formulations, different temperatures ought to be generated during the foaming operation, and accordingly the influence of the temperature in the foam on emissions investigated.

### Performance Tests

The foams produced were rated on the basis of the following physical properties:
a) Foam settling after the end of the rise phase (=fall-back):
   The fall-back, or the further rise, is found from the difference in the foam height after direct blow-off and after 3 minutes after foam blow-off. The foam height is measured at the maximum in the middle of the foam crest by means of a needle secured to a centimeter scale. A negative value here describes the settling of the foam after blow-off; a positive value correspondingly describes the further rise of the foam.
b) Foam height is the height of the freely risen foam formed after 3 minutes. Foam height is reported in centimeters (cm).
c) Rise time
   The period of time between the end of mixing of the reaction components and the blow-off of the polyurethane foam.
d) Density
   The determination is made, as described in DIN EN ISO 845:2009-10, by measurement of the apparent density. The density is reported in kg/m³.
e) Porosity
   The permeability of the foam was determined in accordance with DIN EN ISO 4638:1993-07 by a dynamic pressure measurement on the foam. The dynamic pressure measured was reported in mm water column, with the lower dynamic pressure values then characterizing the more open foam. The values were measured in the range from 0 to 300 mm. The dynamic pressure was measured by means of an apparatus comprising a nitrogen source, a reducing valve with manometer, a screw-thread flow regulator, a wash bottle, a flow meter, a T-piece, a nozzle head and a scaled glass tube filled with water. The applicator nozzle has an edge length of 100×100 mm, a weight of 800 g, a clear width of 5 mm for the outlet hole, a clear width of 20 mm for the lower applicator ring and an outer diameter of 30 mm for the lower applicator ring.
   The measurement is effected by adjusting the nitrogen supply pressure to 1 bar with the reducing valve and adjusting the flow rate to 480 l/h. The amount of water in the scaled glass tube is adjusted such that no pressure differential is built up and none can be read off. For the analysis of the test specimen having dimensions of 250×250×50 mm, the nozzle head is placed onto the corners of the test specimen, flush with the edges, and once onto the (estimated) middle of the test specimen (in each case on the side with the greatest surface area). The result is read off when a constant dynamic pressure has been established.
   Evaluation is effected by forming the average of the five measurements obtained.

### Measurement of Foam Emissions (VOC and Fog Value) Based on Test Method VDA 278 in the Version Dated October 2011:

The method is used to ascertain emissions from non-metallic materials which are employed for moldings within motor vehicles. The emission of volatile organic compounds (VOC value, 30 minutes at 90° C.) and also the fraction of condensable substances (fog value, 60 minutes at 120° C.) was determined in accordance with testing protocol VDA 278 in the version of October 2011. Described below is the procedure for the corresponding thermodesorption with subsequent gas chromatography/mass spectrometry coupling (GC/MS).
a) Measurement technique: The thermal desorption was conducted with a “TDS2” thermal desorber with autosampler from Gerstel, Mülheim, in conjunction with an Agilent 7890/5975 GC/MSD system.
b) Measurement conditions for VOC measurements are reported in tables 4 and 5.

TABLE 4
Thermal desorption analysis parameters for the VOC analysis run

| | |
|---|---|
| Thermal desorption | Gerstel TDS2 |
| Desorption temperature | 90° C. |
| Desorption time | 30 min |
| Flow rate | 65 ml/min |
| Transfer line | 280° C. |
| Cryofocusing | KAS 4 |
| Liner | glass evaporator tube with silanized |
| | glass wool |
| Temperature | −150° C. |

TABLE 5
Gas chromatography-mass spectrometry analysis parameters for the VOC
analysis run

| | |
|---|---|
| GC | capillary - GC Agilent 7890 |
| Injector | PTV split 1:50 |
| Temperature programme | −150° C.; 1 min; [symbol] 10° C./s; 280° C. |
| Column | Agilent 19091B-115, Ultra 2, 50 m * |
| | 0.32 mm FT 0.5 μm |
| Flow rate | 1.3 ml/min const. flow |
| Temperature programme | 50° C.; 2 min; [symbol] 3° C./min; 92° C.; |
| | [symbol] 5° C./min; 160° C.; [symbol] 10° C./min; 280° C., |
| | 20 min |
| Detector | Agilent MSD 5975 |
| Mode | Scan 29-350 amu 2.3 scans/sec |
| Evaluation | Evaluation of the total ion current |
| | chromatogram by calculation as toluene |
| | equivalent |

c) Calibration: For calibration, 2 μl of a mixture of toluene and hexadecane in methanol (each at 0.125 mg/ml) were introduced into a cleaned adsorption tube packed with Tenax® TA (mesh 35/60) and measured (desorption 5 min; 280° C.).
d) Tenax TA is a porous polymer resin based on 2,6-diphenylene oxide, obtainable, for example, from Scientific Instrument Services, 1027 Old York Rd., Ringoes, N.J. 08551.
e) Sample preparation for VOC measurement: 15 mg of foam were positioned in three sample portions in a thermal desorption tube. Care was taken not to compress the foam.
f) Sample preparation for fog measurement: The same foam sample was used as for the VOC analysis. With regard to the measurement arrangement, the VOC analysis was always conducted first and the fog analysis thereafter, ensuring a constant separation between each of the corresponding VOC and fog analyses by means of an autosampler system.
g) The fog measurement conditions are shown in tables 6 and 7.

TABLE 6
Thermal desorption analysis parameters for the fog analysis run

| | |
|---|---|
| Thermal desorption | Gerstel TDS2 |
| Desorption temperature | 120° C. |
| Desorption time | 60 min |
| Flow rate | 65 ml/min |
| Transfer line | 280° C. |
| Cryofocusing | KAS 4 |
| Liner | glass evaporator tube with silanized |
| | glass wool |
| Temperature | −150° C. |

TABLE 7
Gas chromatography-mass spectrometry
analysis parameters for the fog analysis run

| | |
|---|---|
| GC | capillary - GC Agilent 7890 |
| Injector | PTV split 1:50 |
| Temperature programme | −150° C.; 1 min; [symbol] 10° C./s; 280° C. |
| Column | Agilent 19091B-115, Ultra 2, 50 m * |
| | 0.32 mm FT 0.5 μm |
| Flow rate | 1.3 ml/min const. flow |
| Temperature programme | 50° C.; 2 min; [symbol] 25° C./min; 160° C.; |
| | [symbol] 10° C./min; 280° C.; 20 min |
| Detector | Agilent MSD 5975 |
| Mode | Scan 29-450 amu 2.3 scans/sec |
| Evaluation | Evaluation of the total ion current |
| | chromatogram by calculation as |
| | hexadecane equivalent |

h) Calibration: For calibration, 2 μl of a mixture of toluene and hexadecane in methanol (each at 0.125 mg/ml) were introduced into a cleaned adsorption tube packed with Tenax® TA (mesh 35/60) and measured (desorption 5 min; 280° C.).

For the measurement of the emissions, the foams used were those prepared according to formulations II and III and using 500 g of polyol.

### Testing of the Anti-Scorch Performance

a) Microwave test
   The foams obtained by using 100 g of polyol by conversion according to formulation I were irradiated, after the liquid mixture had been poured into the paper-lined wooden boxes, for three minutes in a microwave oven at 1000 W for 80 seconds. The foams were then slit open vertically in the center and the core discoloration was appraised visually. Slight core discoloration was rated +, moderate core discoloration ++, and severe core discoloration +++. A rating of − was given for no perceptible discoloration.
b) Oven test
   The foams obtained using 300 g of polyol by conversion according to formulation I were placed in a drying cabinet at 150° C. for 5 minutes, 3 minutes after the end of the rise time. The paper was then removed and the foam was heated at 180° C. for a further 2 hours. First of all a 3 cm layer is cut off from the bottom. The core discoloration is assessed visually in the subsequent 5 cm layer. Slight core discoloration was rated +, moderate core discoloration ++, and severe core discoloration +++. A rating of − was given for no perceptible discoloration.

### Odor Testing of the Resulting Foams

The completed foams, prepared from 300 g of polyol according to formulation I, were packed in odor-neutral plastic bags and stored in an airtight manner. For the odor assessment of the foam, cubes measuring 10 cm×10 cm×10 cm were cut out and transferred to jars with a volume of 1 l, from which the samples were smelled. The jars were closed with a screw lid. The odor test took place after storing the jars for 24 hours at 22° C.

The odor test was assessed by a panel of 10 trained odor testers. They were questioned here about the intensity of the odor; a low odor level was rated +, moderate odor ++, and high odor +++.

### Results of the Foaming Operations

The inventive additives of Examples 1 and 2, the non-inventive additive described in Example 3, and the commercially available antioxidant mixture ORTEGOL® AO 5 from Evonik Industries AG were tested for their property of inhibiting core discoloration during the foaming operation, in formulation I, and the resulting foams as described above were either microwave-irradiated or heated in an oven. The foams were then cut open and the core discoloration was assessed visually.

In relation to the emissions, the inventive additives of Examples 1 and 2, the non-inventive additive described in Example 3, and the commercially available antioxidant mixture ORTEGOL® AO 5 from Evonik Industries AG were foamed in formulations II and III, and the VOC and fog emissions were determined as described above according to VDA 278 (October 2011).

The results are reproduced in Tables 8-11 below.

As shown in Table 8, without the use of an antioxidant, formulation I gives rise to flexible polyurethane foams which exhibit severe core discoloration (Table 8, entry 1) in both scorching tests (microwave and oven tests). When using 1 pphp of the comparative antioxidant mixture ORTEGOL® AO 5 from Evonik Industries AG, the core discoloration observed was moderate in the microwave test and slight in the oven test (Table 8, entry 2). The non-inventive antioxidant mixture, prepared according to Example 3, likewise yielded flexible polyurethane foams having moderate core discoloration in the microwave test and slight core discoloration in the oven test (Table 8, entry 5). By using 1 pphp of the inventive antioxidant mixtures (Examples 1 and 2), improved discoloration values were observable both in the microwave test and in the oven test (Table 8, entries 3 and 4). Foams characterized by the use of 1 pphp of the antioxidant mixtures ORTEGOL® AO 5 or the non-inventive antioxidant mixture, prepared according to Example 3, have extremely high emission values, whereas foams prepared by using 1 pphp of the inventive antioxidant mixtures according to Example 1 and 2 exhibited very low VOC and fog emission values. These foams were produced according to formulation II (3 pphp H₂O) or according to formulation III (5 pphp H₂O) and the emissions were determined in accordance with VDA 278. No significant differences were apparent for the individual antioxidant mixtures in the two different formulations, and so the effect of different temperatures during the foaming operation on the emissions characteristics can be considered to be negligible. It was nevertheless possible to show that the additive mixtures of the invention, both in the VOC area and in the fog area, exhibited far lower emissions (Table 9, entries 7 and 8; Table 10, entries 11 and 12) than the commercially available antioxidant mixture ORTEGOL® AO 5 and the non-inventive antioxidant mixture prepared in Example 3 (Table 9, entries 6 and 9; Table 10, entries 10 and 13).

As Table 11 shows, the intensity of the odor of the foams produced using the inventive additives from Examples 1 and 2 (entries 15-16) was consistently assessed as being lower than the odor of the foam produced with the comparative antioxidant mixture ORTEGOL® AO 5 from Evonik Industries AG (entry 14). Similarly, the foam produced with the antioxidant mixture according to Example 3 (not inventive) had a stronger odor than the foams produced with the two inventive antioxidant mixtures. The odor test was repeated twice more by the testers, and the aforementioned results were confirmed in precisely the same way. From the results it is evident that the testers assessed a foam treated with one of the additive mixtures of the invention as having a less intense odor.

TABLE 8
Foaming results and core discoloration when using different antioxidants according to formulation I

| | | | | Rise | Fall- | | | Core | Core |
|---|---|---|---|---|---|---|---|---|---|
| | | Amount used | Rise | height | back | Porosity | Density | discoloration, | discoloration, |
| No. | Additive | [pphp] | time [s] | [cm] | [cm] | [mm] | [kg/m³] | microwave test | oven test |
| 1 | Reference | 0 | 96 | 21.5 | 0.2 | 9 | 20.7 | +++ | +++ |
| 2 | ORTEGOL ® AO 5^{a)} | 1 | 94 | 20.3 | 0.1 | 10 | 21.2 | ++ | + |
| 3 | Ex. 1^{b)} | 1 | 98 | 21.0 | 0.2 | 8 | 21.1 | + | − |
| 4 | Ex. 2^{b)} | 1 | 98 | 21.3 | 0.2 | 8 | 21.0 | + | − |
| 5 | Ex. 3^{c)} | 1 | 96 | 21.0 | 0.2 | 7 | 21.1 | ++ | + |

^{a)}Comparative antioxidant mixture from Evonik Industries AG
^{b)}inventive additives prepared according to Examples 1 and 2
^{c)}Non-inventive additive, prepared according to Example 3
− no discoloration apparent
+ slight discoloration
++ moderate discoloration
+++ severe discoloration

TABLE 9
Foaming results and VOC and fog emissions when using different antioxidants according to formulation II

| | | | | | Fall- | | | | |
|---|---|---|---|---|---|---|---|---|---|
| | | Amount used | Rise time | Rise | back | Porosity | Density | VOC | Fog |
| No. | Additive | [pphp] | [s] | height [cm] | [cm] | [mm] | [kg/m³] | [μg/m³] | [μg/m³] |
| 6 | ORTEGOL ® AO 5^{a)} | 1 | 128 | 32.0 | 0.3 | 9 | 29.9 | 61 | 2030 |
| 7 | Ex. 1^{b)} | 1 | 126 | 31.8 | 0.1 | 12 | 30.3 | 29 | 200 |
| 8 | Ex. 2^{b)} | 1 | 126 | 31.7 | 0.2 | 16 | 30.2 | 30 | 171 |
| 9 | Ex. 3^{c)} | 1 | 124 | 32.0 | 0.2 | 13 | 30.1 | 41 | 1467 |

^{a)}Comparative antioxidant mixture from Evonik Industries AG
^{b)}inventive additives prepared according to Examples 1 and 2
^{c)}Non-inventive additive, prepared according to Example 3

TABLE 10
Foaming results and VOC and fog emissions when using different antioxidants according to formulation III

| | | | | | Fall- | | | | |
|---|---|---|---|---|---|---|---|---|---|
| | | Amount used | Rise time | Rise | back | Porosity | Density | VOC | Fog |
| No. | Additive | [pphp] | [s] | height [cm] | [cm] | [mm] | [kg/m³] | [μg/m³] | [μg/m³] |
| 10 | ORTEGOL ® AO 5^{a)} | 1 | 82 | 31.2 | 0.1 | 12 | 17.0 | 63 | 2022 |
| 11 | Ex. 1^{b)} | 1 | 83 | 30.8 | 0.2 | 13 | 17.3 | 30 | 181 |
| 12 | Ex. 2^{b)} | 1 | 81 | 30.9 | 0.1 | 15 | 17.2 | 30 | 145 |
| 13 | Ex. 3^{c)} | 1 | 80 | 31.0 | 0.1 | 10 | 17.0 | 43 | 1411 |

^{a)}Comparative antioxidant mixture from Evonik Industries AG
^{b)}inventive additives prepared according to Examples 1 and 2
^{c)}Non-inventive additive, prepared according to Example 3

TABLE 11
Odor testing of the foams according to
formulation I by 10 trained olfactory testers

| | | | Intensity of the odor | | |
|---|---|---|---|---|---|
| No. | Additive | Amount used [pphp] | +++ | ++ | + |
| 14 | ORTEGOL ® AO 5^{a)} | 1 | 2 | 6 | 2 |
| 15 | Ex. 1^{b)} | 1 | 0 | 3 | 7 |
| 16 | Ex. 2^{b)} | 1 | 1 | 4 | 5 |
| 17 | Ex. 3^{c)} | 1 | 1 | 6 | 3 |

^{a)}Comparative antioxidant mixture from Evonik Industries AG
^{b)}Inventive additives, prepared according to Examples 1 and 2
^{c)}Non-inventive additive, prepared according to Example 3
+ slight odor
++ moderate odor
+++ strong odor

What is claimed is:

1. A compound of the formula (I) 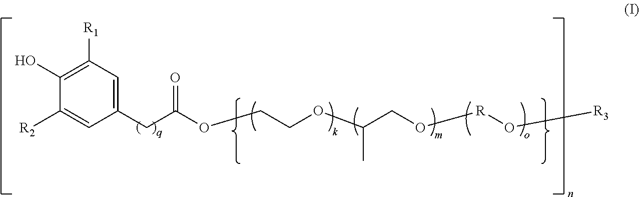
in which
R is CH₂—CH(R^{I}), CH(R^{II})—CH(R^{II}), CH₂—C(R^{II})₂, C(R^{II})₂—C(R^{II})₂, 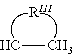
CH₂—CH—CH₂—R^{IV}, C₆H₆—CH—CH₂, or C₆H₆—C(CH₃)—CH₂, where
R^{I} is C₂ to C₂₄ alkyl radical or alkene radical, which may be linear or branched
R^{II} is C₂ to C₂₄ alkyl radical or alkene radical, which may be linear or branched
R^{III} is C₃ to C₆ alkyl radical, which is arranged linearly, and
R^{IV} is OH, Cl, OCH₃, OCH₂—CH₃, O—CH₂—CH═CH₂, O—CH═CH₂, molecule residue of singly or multiply epoxidized fats or oils as mono-, di-, and triglycerides, or molecule residue of singly or multiply epoxidized fatty acids or their C₁-C₂₄ alkyl esters,
R₁ and R₂ independently of one another are straight-chain or branched C₁-C₈ alkyl, cyclopentyl or cyclohexyl, especially tert-butyl,
q is 1, 2 or 3,
n is an integer from 1 to 30,
R₃ is an n-valent radical of linear or branched C₁-C₃₀-alkyl, interrupted in each case optionally by one or more oxygen atoms, or (especially where n=1-12) is an n-valent radical of C₅-C₁₂ cycloalkyl, or a radical R₄ [NR₅—C_{q}H_{2q}—]ₚ,
R₄ is hydrogen, an n-valent radical of linear or branched C₁-C₃₀ alkyl, which is optionally interrupted by one or more groups —NR₅— or (where n=1-12) is an n-valent radical of C₅-C₁₂ cycloalkyl,
R₅ independently at each occurrence is hydrogen or methyl or —C_{q}H_{2q}, and
p corresponds to the number of —[NR₅—C_{q}H_{2q}—] groups that produces n radicals —C_{q}H_{2q}— per molecule,
k is an integer between 0 and 50,
m is an integer between 0 and 50, and
o is an integer between 0 and 50,
where (k+m+o)>10.

2. An antioxidant mixture comprising at least one compound of the formula (I) and a further antioxidant.

3. An antioxidant mixture according to claim 2, comprising as further antioxidant:
at least one benzofuranone derivative of the formula (II) 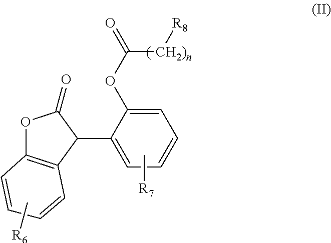
in which
n is an integer between 0 and 7,
R₆ and R₇ independently of one another are H or C₁-C₈ alkyl, 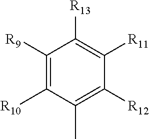
R₈ is H or an aromatic radical where
R₉ and R₁₀ independently of one another are H or C₁-C₆ alkyl, with not both being a C₁-C₆ alkyl,
R₁₁ and R₁₂ independently of one another are H or C₁-C₆ alkyl, with not both being a C₁-C₆ alkyl,
R₁₃ is H or OH.

4. The antioxidant mixture according to claim 3, characterized in that the compound of the formula (I) is present in an amount of 75 to 99 wt % and the compound of the formula (II) is present in an amount of 1 to 25 wt %, wt % being based on the total weight of the compounds of the formulae (I) and (II) used.

5. The antioxidant mixture according to claim 3, further comprising a phosphite of the formula (III), 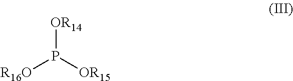
in which
R₁₄, R₁₅ and R₁₆ independently of one another are an aromatic or aliphatic, linear or branched radical of C₁-C₃₀ alkyl or C₂-C₃₀ alkylene, interrupted in each case optionally by one or more oxygen atoms,
the phosphite being present preferably in amounts of 0.1 to 20 wt %, wt % being based on the total weight of the compounds of the formulae (I), (II) and phosphite used.

6. A process for producing polyurethane systems by reaction of at least one polyol component with at least one isocyanate component in the presence of one or more catalysts which catalyse the isocyanate-polyol and/or isocyanate-water reactions and/or the isocyanate trimerization, characterized in that the reaction is carried out in the presence of one or more compounds of the formula (I) or in the presence of an antioxidant mixture according to claim 3.
